(19) United States
(12) Patent Application Publication
(10) Pub. No.: US 2026/0284051 A1
(43) Pub. Date: Sep. 24, 2026

(54) METHIONINE ADENOSYLTRANSFERASE 2A (MAT2A) INHIBITOR COMBINATIONS AND USES THEREOF

(71) Applicant: INSILICO MEDICINE IP LIMITED, Hong Kong (CN)

(72) Inventors: Zhongying CAO, Shanghai (CN); Man ZHANG, Shanghai (CN); Xin CAI, Shanghai (CN); Chiachun CHEN, Shanghai (CN); Xiao DING, Shanghai (CN); Xiaosong LIU, Shanghai (CN); Qingyuan MENG, Shanghai (CN); Feng REN, Shanghai (CN); Hailong WANG, Shanghai (CN)

(21) Appl. No.: 19/476,517

(22) PCT Filed: Apr. 18, 2024
(86) PCT No.: PCT/CN2024/088595

§ 371 Date: Oct. 17, 2025

(30) Foreign Application Priority Data
Apr. 19, 2023 (WO) PCT/CN2023/089330

(51) Int. Cl. A61K 31/517; A61K 31/166; A61K 31/282; A61K 31/337; A61K 31/436; A61K 31/475; A61K 31/496; A61K 31/502; A61K 31/5025; A61K 31/506; A61K 31/519; A61K 31/53; A61K 31/5377; A61K 31/635; A61K 31/7048; A61K 31/706; A61P 35/00

(52) U.S. Cl. CPC A61K 31/517; A61K 31/166; A61K 31/282; A61K 31/337; A61K 31/436; A61K 31/475; A61K 31/496; A61K 31/502; A61K 31/5025; A61K 31/506; A61K 31/519; A61K 31/53; A61K 31/5377; A61K 31/635; A61K 31/7048; A61K 31/706; A61P 35/00

(57) ABSTRACT

Described herein are methods of treating cancer using a small molecule methionine adenosyltransferase 2a (MAT2A) inhibitor and an additional agent.

### CROSS-REFERENCE

This patent application claims the benefit of International Application No. PCT/CN2023/089330, filed Apr. 19, 2023, which is incorporated herein by reference in its entirety.

### BACKGROUND

Methionine adenosyltransferase 2a (MAT2A) plays an important role in metabolism and epigenetics. Despite its broad cellular role, inhibition of MAT2A has been shown to result in a selective anti-proliferative effect in cancers with deletion of a separate metabolic gene, methylthioadenosine phosphorylase (“MTAP”). MTAP deficiency occurs frequently in both solid tumors and hematologic malignancies. As such, compounds that inhibit MAT2A are potential agents for treating MTAP-deficient cancers.

### SUMMARY

Disclosed herein is a method of treating cancer in a subject in need thereof, the method comprising administering to the subject:
(a) a compound of Formula (I) or Formula (II), or a pharmaceutically acceptable salt thereof:

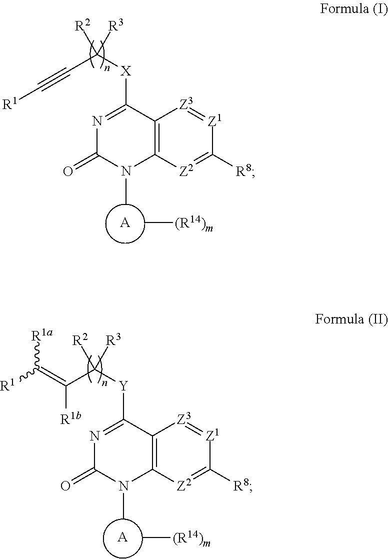

and
(b) an additional agent,
   wherein the combined amount of the compound of Formula (I) or Formula (II), or a pharmaceutically acceptable salt thereof and the additional agent is therapeutically effective for treating the cancer.

Also disclosed herein is a method of treating cancer in a subject in need thereof, the method comprising administering to the subject:
(a) 5-methoxy-1-(2-methylpyridin-3-yl)-4-(prop-2-yn-1-ylamino)-7-(trifluoromethyl)quinazolin-2(1H)-one:

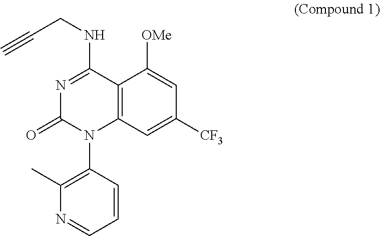 [SMILES: C#CCNc1nc(=O)n(-c2cccnc2C)c2cc(C(F)(F)F)cc(OC)c12]

or a pharmaceutically acceptable salt thereof;
and
(b) an additional agent.

In some embodiments, the cancer is a primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the cancer is liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.

In some embodiments, the additional agent is a PARP inhibitor, a CHK1 inhibitor, a MDM2 inhibitor, a hypomethylating agent, an mTOR inhibitor, an ATM inhibitor, a CDK 4/6 inhibitor, a BCL-2 inhibitor, a PRMT5 inhibitor, a PRMT1 inhibitor, an ATR inhibitor, a WEE1 inhibitor, an APE1 inhibitor, a topoisomerase inhibitor, a taxane, an immune checkpoint inhibitor, a CDK7 inhibitor, a CDK9 inhibitor, a DNA synthesis inhibitor, an antimetabolite, an AURORA inhibitor, a microtubule stabilizer, a DNA cross-linker, a vinca alkaloid, an alkylating agent, a PRMT6 inhibitor, a PRMT7 inhibitor, a PRMT9 inhibitor, a KRAS inhibitor, an EGFR inhibitor, a VEGFR inhibitor, an aromatase inhibitor, a mitotic inhibitor, a radiopharmaceutical agent, a cytotoxic agent, or any combination thereof.

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows the efficacy of Compound 1 and Docetaxel in KP4 MTAP null xenografts.

FIG. 2 shows the mice body weight change rate.

FIG. 3 shows the efficacy of Compound 1 and Docetaxel in HCC15 MTAP null xenografts.

FIG. 4 shows the mice body weight change rate.

FIG. 5 shows the efficacy of Compound 1 and MRTX1719 in NCI-H838 MTAP null xenografts.

FIG. 6 shows the mice body weight change rate.

FIG. 7A shows Excess over Bliss synergy of Compound 1 and PARP inhibitor olaparib combinations in a MTAP-deficient cell line (RT112-84).

FIG. 7B shows Excess over Bliss synergy of Compound 1 and PARP inhibitor olaparib combinations in a MTAP-deficient cell line (DOHH-2).

FIG. 7C shows Excess over Bliss synergy of Compound 1 and PARP inhibitor olaparib combinations in a MTAP-deficient cell line (Jurkat).

FIG. 7D shows Excess over Bliss synergy of Compound 1 and PARP inhibitor olaparib combinations in a MTAP-deficient cell line (HuP-T4).

FIG. 8A shows Excess over Bliss synergy of Compound 1 and PARP inhibitor niraparib combinations in MTAP-deficient cell line (RT112-84).

FIG. 8B shows Excess over Bliss synergy of Compound 1 and PARP inhibitor niraparib combinations in a MTAP-deficient cell line (DOHH-2).

FIG. 8C shows Excess over Bliss synergy of Compound 1 and PARP inhibitor niraparib combinations in a MTAP-deficient cell line (HuP-T4).

FIG. 9A shows Excess over Bliss synergy of Compound 1 and PARP inhibitor talazoparib combinations in a MTAP-deficient cell line (CAPAN-1).

FIG. 9B shows Excess over Bliss synergy of Compound 1 and PARP inhibitor talazoparib combinations in a MTAP-deficient cell line (Jurkat).

FIG. 9C shows Excess over Bliss synergy of Compound 1 and PARP inhibitor talazoparib combinations in a MTAP-deficient cell line (SW1573).

FIG. 9D shows Excess over Bliss synergy of Compound 1 and PARP inhibitor talazoparib combinations in a MTAP-deficient cell line (HuP-T4).

FIG. 10A shows Excess over Bliss synergy of Compound 1 and PARP inhibitor rucaparib combinations in a MTAP-deficient cell line (DOHH-2).

FIG. 10B shows Excess over Bliss synergy of Compound 1 and PARP inhibitor rucaparib combinations in a MTAP-deficient cell line (RS4-11).

FIG. 11A shows Excess over Bliss synergy of Compound 1 and BCL-2 inhibitor venetoclax combinations in a MTAP-deficient cell line (RT112-84).

FIG. 11B shows Excess over Bliss synergy of Compound 1 and BCL-2 inhibitor venetoclax combinations in a MTAP-deficient cell line (THP-1).

FIG. 11C shows Excess over Bliss synergy of Compound 1 and BCL-2 inhibitor venetoclax combinations in a MTAP-deficient cell line (DOHH-2).

FIG. 11D shows Excess over Bliss synergy of Compound 1 and BCL-2 inhibitor venetoclax combinations in a MTAP-deficient cell line (Jurkat).

FIG. 11E shows Excess over Bliss synergy of Compound 1 and BCL-2 inhibitor venetoclax combinations in a MTAP-deficient cell line (SW780).

FIG. 11F shows Excess over Bliss synergy of Compound 1 and BCL-2 inhibitor venetoclax combinations in a MTAP-deficient cell line (LN-18).

FIG. 12A shows Excess over Bliss synergy of Compound 1 and EGFR inhibitor afatinib combinations in a MTAP-deficient cell line (RT112-84).

FIG. 12B shows Excess over Bliss synergy of Compound 1 and EGFR inhibitor afatinib combinations in a MTAP-deficient cell line (DOHH-2).

FIG. 12C shows Excess over Bliss synergy of Compound 1 and EGFR inhibitor afatinib combinations in a MTAP-deficient cell line (HCC1395).

FIG. 12D shows Excess over Bliss synergy of Compound 1 and EGFR inhibitor afatinib combinations in a MTAP-deficient cell line (Jurkat).

FIG. 12E shows Excess over Bliss synergy of Compound 1 and EGFR inhibitor afatinib combinations in a MTAP-deficient cell line (HCT-116 MTAP-null).

FIG. 12F shows Excess over Bliss synergy of Compound 1 and EGFR inhibitor afatinib combinations in a MTAP-deficient cell line (A549).

FIG. 13A shows Excess over Bliss synergy of Compound 1 and EGFR inhibitor gefitinib combinations in a MTAP-deficient cell line (DOHH-2).

FIG. 13B shows Excess over Bliss synergy of Compound 1 and EGFR inhibitor gefitinib combinations in a MTAP-deficient cell line (HCC1395).

FIG. 13C shows Excess over Bliss synergy of Compound 1 and EGFR inhibitor gefitinib combinations in a MTAP-deficient cell line (Jurkat).

FIG. 13D shows Excess over Bliss synergy of Compound 1 and EGFR inhibitor gefitinib combinations in a MTAP-deficient cell line (LN-18).

FIG. 13E shows Excess over Bliss synergy of Compound 1 and EGFR inhibitor gefitinib combinations in a MTAP-deficient cell line (HuP-T4).

FIG. 14A shows Excess over Bliss synergy of Compound 1 and EGFR inhibitor erlotinib combinations in a MTAP-deficient cell line (LN-18).

FIG. 14B shows Excess over Bliss synergy of Compound 1 and EGFR inhibitor erlotinib combinations in a MTAP-deficient cell line (SK-HEP-1).

FIG. 14C shows Excess over Bliss synergy of Compound 1 and EGFR inhibitor erlotinib combinations in a MTAP-deficient cell line (A549).

FIG. 15A shows Excess over Bliss synergy of Compound 1 and mTOR inhibitor everolimus combinations in a MTAP-deficient cell line (CAPAN-1).

FIG. 15B shows Excess over Bliss synergy of Compound 1 and mTOR inhibitor everolimus combinations in a MTAP-deficient cell line (HCT-116 MTAP-null).

FIG. 15C shows Excess over Bliss synergy of Compound 1 and mTOR inhibitor everolimus combinations in a MTAP-deficient cell line (NCI-H1651).

FIG. 15D shows Excess over Bliss synergy of Compound 1 and mTOR inhibitor everolimus combinations in a MTAP-deficient cell line (BT-474).

FIG. 16A shows Excess over Bliss synergy of Compound 1 and mTOR inhibitor temsirolimus combinations in a MTAP-deficient cell line (Jurkat).

FIG. 16B shows Excess over Bliss synergy of Compound 1 and mTOR inhibitor temsirolimus combinations in a MTAP-deficient cell line (MDA-MB-231).

FIG. 16C shows Excess over Bliss synergy of Compound 1 and mTOR inhibitor temsirolimus combinations in a MTAP-deficient cell line (BT-474).

FIG. 17A shows Excess over Bliss synergy of Compound 1 and KRAS inhibitor sotorasib combinations in a MTAP-deficient cell line (DOHH-2).

FIG. 17B shows Excess over Bliss synergy of Compound 1 and KRAS inhibitor sotorasib combinations in a MTAP-deficient cell line (SW1573).

FIG. 17C shows Excess over Bliss synergy of Compound 1 and KRAS inhibitor sotorasib combinations in a MTAP-deficient cell line (MIA-Paca-2).

FIG. 17D shows Excess over Bliss synergy of Compound 1 and KRAS inhibitor sotorasib combinations in a MTAP-deficient cell line (RS4-11).

FIG. 17E shows Excess over Bliss synergy of Compound 1 and KRAS inhibitor sotorasib combinations in a MTAP-deficient cell line (SW1088).

FIG. 17F shows Excess over Bliss synergy of Compound 1 and KRAS inhibitor sotorasib combinations in a MTAP-deficient cell line (HCT-116-MTAP-null).

FIG. 17G shows Excess over Bliss synergy of Compound 1 and KRAS inhibitor sotorasib combinations in a MTAP-deficient cell line (A549).

FIG. 17H shows Excess over Bliss synergy of Compound 1 and KRAS inhibitor sotorasib combinations in a MTAP-deficient cell line (HuP-T4).

FIG. 18A shows Excess over Bliss synergy of Compound 1 and WEE1 inhibitor bosutinib combinations in a MTAP-deficient cell line (Jurkat).

FIG. 18B shows Excess over Bliss synergy of Compound 1 and WEE1 inhibitor bosutinib combinations in a MTAP-deficient cell line (LN-18).

FIG. 18C shows Excess over Bliss synergy of Compound 1 and WEE1 inhibitor bosutinib combinations in a MTAP-deficient cell line (RS4-11).

FIG. 18D shows Excess over Bliss synergy of Compound 1 and WEE1 inhibitor bosutinib combinations in a MTAP-deficient cell line (HCT-116-MTAP-null).

FIG. 18E shows Excess over Bliss synergy of Compound 1 and WEE1 inhibitor bosutinib combinations in a MTAP-deficient cell line (A549).

FIG. 19A shows Excess over Bliss synergy of Compound 1 and CDK4/6 inhibitor palbociclib combinations in a MTAP-deficient cell line (DOHH-2).

FIG. 19B shows Excess over Bliss synergy of Compound 1 and CDK4/6 inhibitor abemaciclib combinations in a MTAP-deficient cell line (UM-UC-3).

FIG. 19C shows Excess over Bliss synergy of Compound 1 and CDK4/6 inhibitor abemaciclib combinations in a MTAP-deficient cell line (SW900).

FIG. 20A shows Excess over Bliss synergy of Compound 1 and topoisomerase inhibitor etoposide combinations in a MTAP-deficient cell line (NCI-H292).

FIG. 20B shows Excess over Bliss synergy of Compound 1 and topoisomerase inhibitor etoposide combinations in a MTAP-deficient cell line (THP-1).

FIG. 20C shows Excess over Bliss synergy of Compound 1 and topoisomerase inhibitor etoposide combinations in a MTAP-deficient cell line (LN-18).

FIG. 20D shows Excess over Bliss synergy of Compound 1 and topoisomerase inhibitor etoposide combinations in a MTAP-deficient cell line (HCC1806).

FIG. 20E shows Excess over Bliss synergy of Compound 1 and topoisomerase inhibitor etoposide combinations in a MTAP-deficient cell line (UM-UC-3).

FIG. 20F shows Excess over Bliss synergy of Compound 1 and topoisomerase inhibitor etoposide combinations in a MTAP-deficient cell line (A172).

FIG. 20G shows Excess over Bliss synergy of Compound 1 and topoisomerase inhibitor etoposide combinations in a MTAP-deficient cell line (SW900).

FIG. 20H shows Excess over Bliss synergy of Compound 1 and topoisomerase inhibitor etoposide combinations in a MTAP-deficient cell line (HCC38).

FIG. 21A shows Excess over Bliss synergy of Compound 1 and antimetabolites pemetrexed combinations in a MTAP-deficient cell line (DOHH-2).

FIG. 21B shows Excess over Bliss synergy of Compound 1 and antimetabolites pemetrexed combinations in a MTAP-deficient cell line (Jurkat).

FIG. 21C shows Excess over Bliss synergy of Compound 1 and antimetabolites pemetrexed combinations in a MTAP-deficient cell line (SW780).

FIG. 21D shows Excess over Bliss synergy of Compound 1 and antimetabolites pemetrexed combinations in a MTAP-deficient cell line (LN-18).

FIG. 21E shows Excess over Bliss synergy of Compound 1 and antimetabolites pemetrexed combinations in a MTAP-deficient cell line (HCC38).

FIG. 21F shows Excess over Bliss synergy of Compound 1 and antimetabolites pemetrexed combinations in a MTAP-deficient cell line (HCC1806).

FIG. 22A shows Excess over Bliss synergy of Compound 1 and hypomethylating agent decitabine combinations in a MTAP-deficient cell line (CAPAN-1).

FIG. 22B shows Excess over Bliss synergy of Compound 1 and hypomethylating agent decitabine combinations in a MTAP-deficient cell line (HuP-T4).

FIG. 23A shows Excess over Bliss synergy of Compound 1 and antimetabolites capecitabine or 5-FU combinations in a MTAP-deficient cell line (NCI-H1651).

FIG. 23B shows Excess over Bliss synergy of Compound 1 and antimetabolites capecitabine or 5-FU combinations in a MTAP-deficient cell line (Jurkat).

FIG. 23C shows Excess over Bliss synergy of Compound 1 and antimetabolites capecitabine or 5-FU combinations in a MTAP-deficient cell line (K562).

FIG. 24A shows Excess over Bliss synergy of Compound 1 and microtubule-stabilizing agent docetaxel combinations in a MTAP-deficient cell line (SK-HEP-1).

FIG. 24B shows Excess over Bliss synergy of Compound 1 and microtubule-stabilizing agent docetaxel combinations in a MTAP-deficient cell line (RT4).

FIG. 24C shows Excess over Bliss synergy of Compound 1 and microtubule-stabilizing agent docetaxel combinations in a MTAP-deficient cell line (PANC-1).

FIG. 25A shows Excess over Bliss synergy of Compound 1 and vinca alkaloid vinorelbine combinations in a MTAP-deficient cell line (RT4).

FIG. 25B shows Excess over Bliss synergy of Compound 1 and vinca alkaloid vinorelbine combinations in a MTAP-deficient cell line (SK-HEP-1).

FIG. 26A shows Excess over Bliss synergy of Compound 1 and alkylating agent oxaliplatin combinations in a MTAP-deficient cell line (Jurkat).

FIG. 26B shows Excess over Bliss synergy of Compound 1 and alkylating agent oxaliplatin combinations in a MTAP-deficient cell line (DOHH-2).

FIG. 26C shows Excess over Bliss synergy of Compound 1 and alkylating agent oxaliplatin combinations in a MTAP-deficient cell line (HuP-T4).

FIG. 27A shows Excess over Bliss synergy of Compound 1 and alkylating agent altretamine combinations in a MTAP-deficient cell line (DOHH-2).

FIG. 27B shows Excess over Bliss synergy of Compound 1 and alkylating agent altretamine combinations in a MTAP-deficient cell line (SW1573).

FIG. 28A shows Excess over Bliss synergy of Compound 1 and hypomethylating agent procainamide combinations in a MTAP-deficient cell line (DOHH-2).

FIG. 28B shows Excess over Bliss synergy of Compound 1 and hypomethylating agent procainamide combinations in a MTAP-deficient cell line (HCC1395).

FIG. 28C shows Excess over Bliss synergy of Compound 1 and hypomethylating agent procainamide combinations in a MTAP-deficient cell line (HCC38).

FIG. 29A shows Excess over Bliss synergy of Compound 1 and PRMT5 inhibitor MRTX1719 combinations in a MTAP-deficient cell line (NCI-H838).

FIG. 29B shows Excess over Bliss synergy of Compound 1 and PRMT5 inhibitor AM-9747 combinations in a MTAP-deficient cell line (NCI-H838).

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-I-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-I-pentyl, 4-methyl-I-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁o fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₅ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁—C heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₆ heterocycloalkenyl), from two to five carbon atoms (C₂-C₅ fully saturated heterocycloalkyl or C₂-C₅ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The terms “effective amount” or “therapeutically effective amount,” as used herein, refer to a sufficient amount of an agent or a compound, or a combination of agents or compounds being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

The term “acceptable” with respect to a formulation, composition or ingredient, as used herein, means having no persistent detrimental effect on the general health of the subject being treated.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “about” means within a statistically meaningful range of a value, such as a stated concentration range, time frame, molecular weight, particle size, temperature, or pH. Such a range can be within an order of magnitude, typically within 10%, more typically within 5%, and even more typically within 3% of the indicated value or range. Sometimes, such a range can be within the experimental error typical of standard methods used for the measurement and/or determination of a given value or range.

The allowable variation encompassed by the term “about” will depend upon the particular system under study, and can be readily appreciated by one of ordinary skill in the art. Whenever a range is recited within this application, every whole number integer within the range is also contemplated as an embodiment of the invention.

As used herein, the term ‘MTAP-deficient cancer’ refers to a cancer which lacks activity of the metabolic enzyme methylthioadenosine phosphorylase (MTAP). Thus, an MTAP-deficient cancer is a cancer that is associated with a failure to express the MTAP gene, which failure may be attributable to the absence of MTAP gene, the lack of MTAP protein expression, or accumulation of MTAP substrate MTA. In some embodiments the term ‘MTAP-deficient’ is referred to as ‘MTAP-deleted’ and/or ‘MTAP-null’ and thus the three terms may be used interchangeably. For example in some embodiments, ‘MTAP-deleted’ or ‘MT AP-null’ cancer refers to chromosomal loss of the MTAP gene, resulting in full or partial loss of MTAP DNA which prevents expression of functional, full length MTAP protein. In some embodiments a MTAP-deficient cancer is a cancer where the locus of the CDKN2A gene is absent or deleted. In some embodiments, an MTAP-deficient cancer is one in which the MTAP gene has been deleted, lost, or otherwise deactivated. In some embodiments, an MTAP-deficient cancer is a cancer in which the MTAP protein has a reduced function or is functionally impaired as compared to a wild type MTAP gene. Accordingly, in an embodiment of the present disclosure, there is provided a method for treating a MTAP-deficient cancer in a subject, wherein the cancer is characterized by at least one of (i) a reduction or absence of MTAP expression; (ii) absence of the MTAP gene; and (iii) reduced function of MTAP protein, as compared to the corresponding cancers where the MTAP gene and/or protein is present and fully functioning, or as compared to the corresponding cancers with the wild type MTAP gene.

As used herein, the term “wild type MTAP cancer” or “MTAP wild type cancer” refers to a cancer in which the activity of the metabolic enzyme methylthioadenosine phosphorylase (MTAP) is intact. Thus, a wild type MTAP cancer is a cancer that expresses the MTAP gene and the MTAP protein.

### MAT2A Inhibitors

In some embodiments disclosed herein is an MAT2A inhibitor of Formula (I), Formula (II), or a pharmaceutically acceptable salt thereof:

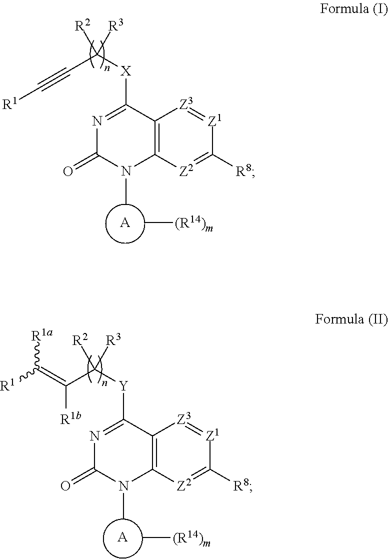

wherein:
\textcircled{A} is selected from C₃₋₆cycloalkyl, C₂₋₉heterocycloalkyl, C₆₋₁₀aryl, and C₁₋₁₀heteroaryl;
Z¹ is CR⁷ or N;
Z² is CR⁹ or N;
Z³ is CR⁶ or N;
Z⁴ is CR^{6a} or N;
X is selected from —N(R⁴)—, —O—, and —C(R⁵)(R^{5a})—;
Y is selected from —N(R^{4a})—, —O—, and —C(R⁵)(R^{5a})—;
R¹ is selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15a};
R^{1a} and R^{1b} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15a};
each R² and each R³ are each independently selected from hydrogen, halogen, oxo, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15b}; or R² and R³, together with the carbon to which they are attached, form a C₃₋₆cycloalkyl or C₂₋₉heterocycloalkyl;
R⁴ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R⁴ and an R³ are combined to form a C₂₋₉heterocycloalkyl optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
R^{4a} is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R^{4a} and an R³ are combined to form a C₂₋₉heterocycloalkyl optionally substituted with one, two, or three groups selected from halogen, —CN, hydroxy, C₁₋₆alkyl, C₁₋₆haloalkyl, C₁₋₆alkoxy, C₃.(cycloalkyl, C₂₋₉heterocycloalkyl, C₆₋₁₀aryl, and C₁₋₉heteroaryl;
R⁵ and R^{5a} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R₁₀)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R⁵ and an R³ are combined to form a C₃₋₆cycloalkyl, C₂₋₉heterocycloalkyl, C₆₋₁₀aryl, or C₂₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
R⁶, R⁷, R⁸, and R⁹ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c};
R^{6a} is selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from R^{15c};
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁴ is independently selected from halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15d};
each R^{15a}, R^{15b}, R^{15c}, and R^{15d} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₁₀heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
m is 0, 1, 2, 3, 4, or 5; and
n is 0, 1, 2, 3, 4, 5, or 6.

In some embodiments disclosed herein is an MAT2A inhibitor of Formula (I), or a pharmaceutically acceptable salt thereof. In some embodiments disclosed herein is an MAT2A inhibitor of Formula (II), or a pharmaceutically acceptable salt thereof.

In some embodiments of a compound of Formula (I), or a pharmaceutically acceptable salt thereof, X is —N(R⁴)—. In some embodiments, X is —N(R⁴)— and R⁴ is selected from hydrogen, C₁₋₆alkyl, C₁₋₆haloalkyl, C₂-,alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, X is —N(R⁴)— and R⁴ is hydrogen or C₁₋₆alkyl. In some embodiments, X is —N(R⁴)— and R⁴ is hydrogen. In some embodiments, X is —N(R⁴)— and R⁴ is C₁₋₆alkyl. In some embodiments, X is —N(R⁴)— and R⁴ is —CH₃. In some embodiments, X is —N(R⁴)— and R⁴ and an R³ are combined to form a C₂₋₉heterocycloalkyl optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, X is —N(R⁴)— and R⁴ and an R³ are combined to form a C₂₋₉heterocycloalkyl optionally substituted with one, two, or three groups selected from halogen, hydroxy, C₁₋₆alkyl, C₁₋₆haloalkyl, and C₁₋₆alkoxy. In some embodiments, X is —N(R⁴)— and R⁴ and an R³ are combined to form a piperidinyl, piperazinyl, pyrrolidinyl, or azetidinyl ring optionally substituted with one, two, or three groups selected from halogen, hydroxy, C₁₋₆alkyl, C₁₋₆haloalkyl, and C₁₋₆alkoxy.

In some embodiments, X is —N(R⁴)— and R⁴ and an R³ are combined to form an unsubstituted piperidinyl, piperazinyl, pyrrolidinyl, or azetidinyl ring.

In some embodiments of a compound of Formula (I), or a pharmaceutically acceptable salt thereof, X is —O—.

In some embodiments of a compound of Formula (I), or a pharmaceutically acceptable salt thereof, X is —C(R⁵)(R^{5a})—. In some embodiments, X is —C(R⁵)(R^{5a})— and R⁵ and R^{5a} are independently selected from hydrogen and C₁₋₆alkyl. In some embodiments, X is —C(R⁵)(R^{5a})— and R⁵ and R^{5a} are hydrogen. In some embodiments, X is —C(R⁵)(R^{5a})— and R⁵ and R^{5a} are C₁₋₆alkyl. In some embodiments, X is —C(R⁵)(R^{5a})—, R⁵ is hydrogen, and R^{5a} is C₁₋₆alkyl.

In some embodiments of a compound of Formula (II), or a pharmaceutically acceptable salt thereof, Y is —N(R^{4a})—. In some embodiments, Y is —N(R^{4a})— and R^{4a} is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl. In some embodiments, Y is —N(R^{4a})— and R^{4a} is hydrogen or C₁₋₆alkyl. In some embodiments, Y is —N(R^{4a})— and R^{4a} is hydrogen. In some embodiments, Y is —N(R^{4a})— and R^{4a} is C₁₋₆alkyl. In some embodiments, Y is —N(R^{4a})— and R^{4a} is —CH₃.

In some embodiments of a compound of Formula (II), or a pharmaceutically acceptable salt thereof, Y is —O—.

In some embodiments of a compound of Formula (II), or a pharmaceutically acceptable salt thereof, Y is —C(R⁵)(R^{5a})—. In some embodiments, Y is —C(R⁵)(R^{5a})— and R⁵ and R^{5a} are independently selected from hydrogen and C₁₋₆alkyl. In some embodiments, Y is —C(R⁵)(R^{5a})— and RS and R^{5a} are hydrogen. In some embodiments, Y is —C(R⁵)(R^{5a})— and R⁵ and R^{5a} are C₁₋₆alkyl. In some embodiments, Y is —C(R⁵)(R^{5a})—, R⁵ is hydrogen, and R^{5a} is C₁₋₆alkyl.

In some embodiments of a compound of Formula (I) or (II), or a pharmaceutically acceptable salt thereof, each R² is independently selected from hydrogen and C₁₋₆alkyl. In some embodiments, or a pharmaceutically acceptable salt thereof, each R² is hydrogen. In some embodiments, or a pharmaceutically acceptable salt thereof, each R³ is independently selected from hydrogen and C₁₋₆alkyl. In some embodiments, each R³ is hydrogen. In some embodiments, each R² and R³, together with the carbon to which they are attached, form a C₃₋₆cycloalkyl. In some embodiments, each R² and R³, together with the carbon to which they are attached, form a cyclopropyl ring. In some embodiments, each R² and R³, together with the carbon to which they are attached, form a cyclobutyl ring. In some embodiments, each R² and R³, together with the carbon to which they are attached, form a cyclopentyl ring. In some embodiments, each R² and R³, together with the carbon to which they are attached, form a cyclohexyl ring.

In some embodiments of a compound of Formula (I) or (II), or a pharmaceutically acceptable salt thereof, n is 1, 2, or 3. In some embodiments, n is 1. In some embodiments, n is 2. In some embodiments, n is 3. In some embodiments, n is 4. In some embodiments, n is 5. In some embodiments, n is 6.

In some embodiments of a compound of Formula (I) or (II), or a pharmaceutically acceptable salt thereof, Z¹ is CR⁷. In some embodiments, Z¹ is CR⁷ and R⁷ is hydrogen, halogen, —CN, C₁₋₆alkyl, C₁₋₆haloalkyl, —OR¹⁰, or —N(R¹⁰)(R¹¹). In some embodiments, Z¹ is CR⁷ and R⁷ is hydrogen, halogen, C₁₋₆alkyl, or C₁₋₆haloalkyl. In some embodiments, Z¹ is CR⁷ and R⁷ is hydrogen.

In some embodiments of a compound of Formula (I) or (II), or a pharmaceutically acceptable salt thereof, Z¹ is N.

In some embodiments of a compound of Formula (I) or (II), or a pharmaceutically acceptable salt thereof, Z² is CR⁹. In some embodiments, Z² is CR⁹ and R⁹ is hydrogen, halogen, —CN, C₁₋₆alkyl, C₁₋₆haloalkyl, —OR¹⁰, or —N(R¹⁰)(R¹¹). In some embodiments, Z² is CR⁹ and R⁹ is hydrogen, halogen, C₁₋₆ alkyl, or C₁₋₆haloalkyl. In some embodiments, Z² is CR⁹ and R⁹ is hydrogen.

In some embodiments of a compound of Formula (I) or (II), or a pharmaceutically acceptable salt thereof, Z² is N.

In some embodiments of a compound of Formula (I), or a pharmaceutically acceptable salt thereof, Z³ is CR⁶. In some embodiments, Z³ is CR⁶ and R⁶ is selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR₁₀, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c}. In some embodiments of a compound of Formula (I), Z³ is CR⁶ and R⁶ is selected from hydrogen, halogen, —CN, C₁₋₆alkyl, C₁₋₆haloalkyl, —OR¹⁰, and —N(R¹⁰)(R¹¹). In some embodiments, Z³ is CR⁶ and R⁶ is selected from hydrogen and —OR¹⁰ and R¹¹ is C₁₋₆alkyl. In some embodiments, Z³ is CR⁶ and R⁶ is hydrogen.

In some embodiments of a compound of Formula (I), or a pharmaceutically acceptable salt thereof, Z³ is N.

In some embodiments of a compound of Formula (II), or a pharmaceutically acceptable salt thereof, Z⁴ is CR^{6a}. In some embodiments of a compound of Formula (II), or a pharmaceutically acceptable salt thereof, Z⁴ is CR^{6a} and R^{6a} is selected from hydrogen, halogen, —CN, C₁₋₆alkyl, C₁₋₆haloalkyl, —OR¹⁰, and —N(R¹⁰)(R¹¹). In some embodiments of a compound of Formula (II), or a pharmaceutically acceptable salt thereof, Z⁴ is CR^{6a} and R^{6a} is selected from hydrogen and —OR¹⁰ and RIO is C₁₋₆alkyl. In some embodiments of a compound of Formula (II), or a pharmaceutically acceptable salt thereof, Z⁴ is CR^{6a} and R^{6a} is hydrogen.

In some embodiments of a compound of Formula (I) or (II), or a pharmaceutically acceptable salt thereof, \textcircled{A} is C₁₋₁₀heteroaryl. In some embodiments, \textcircled{A} is C₁₋₁₀heteroaryl selected from pyridyl, pyrimidyl, pyrazinyl, and pyridazinyl. In some embodiments, \textcircled{A} is 5 or 6 membered heteroaryl. In some embodiments, \textcircled{A} is 5 membered heteroaryl. In some embodiments, \textcircled{A} is 6 membered heteroaryl. In some embodiments, \textcircled{A} is pyridyl. In some embodiments, \textcircled{A} is pyrimidyl. In some embodiments, \textcircled{A} is pyrazinyl. In some embodiments, \textcircled{A} is pyridazinyl.

In some embodiments of a compound of Formula (I) or (II), or a pharmaceutically acceptable salt thereof, \textcircled{A} is C₆₋₁₀aryl. In some embodiments, \textcircled{A} is phenyl.

In some embodiments of a compound of Formula (I) or (II), or a pharmaceutically acceptable salt thereof, \textcircled{A} is C₂₋₉heterocycloalkyl.

In some embodiments of a compound of Formula (I) or (II), or a pharmaceutically acceptable salt thereof, \textcircled{A} is C₃₋₆cycloalkyl.

In some embodiments of a compound of Formula (I) or (II), or a pharmaceutically acceptable salt thereof, each R¹⁴ is independently selected from halogen, C₁₋₆alkyl, C₁₋₆haloalkyl, —OR¹⁰, and —N(R¹⁰)(R¹¹). In some embodiments, each R¹⁴ is independently selected from halogen and C₁₋₆alkyl. In some embodiments, each R¹⁴ is independently selected from halogen. In some embodiments, each R¹⁴ is independently selected from C₁₋₆alkyl.

In some embodiments of a compound of Formula (I) or (II), or a pharmaceutically acceptable salt thereof, m is 0, 1, or 2. In some embodiments, m is 0 or 1. In some embodiments, m is 0. In some embodiments, m is 1. In some embodiments, m is 2. In some embodiments, m is 3.

In some embodiments of a compound of Formula (I) or (II), or a pharmaceutically acceptable salt thereof, R⁸ is selected from halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c}.

In some embodiments of a compound of Formula (I) or (II), or a pharmaceutically acceptable salt thereof, R⁸ is hydrogen, halogen, —CN, C₁₋₆alkyl, C₁₋₆haloalkyl, —OR¹⁰, or C₃₋₆cycloalkyl. In some embodiments, R⁸ is hydrogen, halogen, or C₁₋₆haloalkyl. In some embodiments, R⁸ is hydrogen. In some embodiments, R⁸ is halogen. In some embodiments, R⁸ is C₁₋₆haloalkyl. In some embodiments, R⁸ is —CF₃. In some embodiments, R⁸ is C₁₋₆alkyl. In some embodiments, R⁸ is —CH₃. In some embodiments of a compound of Formula (I), or a pharmaceutically acceptable salt thereof, R⁸ is C₃₋₁₀cycloalkyl. In some embodiments, R⁸ is cyclopropyl. In some embodiments, R⁸ is —CN.

In some embodiments of a compound of Formula (I) or (II), or a pharmaceutically acceptable salt thereof, R¹ is selected from hydrogen, C₁₋₆alkyl, C₁₋₆haloalkyl, C₃₋₆cycloalkyl, C₂₋₉heterocycloalkyl, and C₁₋₉heteroaryl, wherein C₁₋₆alkyl, C₃₋₆cycloalkyl, C₂₋₉heterocycloalkyl, and C₁₉heteroaryl are optionally substituted with one, two, or three groups selected from R^{15a}.

In some embodiments of a compound of Formula (I) or (II), or a pharmaceutically acceptable salt thereof, R¹ is hydrogen.

In some embodiments of a compound of Formula (I) or (II), or a pharmaceutically acceptable salt thereof, R¹ is C₁₋₆alkyl optionally substituted with one, two, or three groups selected from Risa. In some embodiments, R¹ is C₁₋₆alkyl optionally substituted with one, two, or three groups selected from C₂₋₉heterocycloalkyl, C₁₋₉heteroaryl, —OR¹⁰, and —N(R¹⁰)(R¹¹). In some embodiments, R¹ is C₁₋₆alkyl substituted with one group selected from C₂₋₉heterocycloalkyl, C₁₋₉heteroaryl, —OR¹⁰, and —N(R¹⁰)(R¹¹). In some embodiments, R¹ is C₁₋₆alkyl substituted with one group selected from C₂₋₉heterocycloalkyl, C₁₋₉heteroaryl, —OR¹⁰, and —N(R¹⁰)(R¹¹) and R¹⁰ and R are independently selected from hydrogen and C₁₋₆alkyl. In some embodiments, R¹ is unsubstituted C₁₋₆alkyl.

In some embodiments of a compound of Formula (I) or (II), or a pharmaceutically acceptable salt thereof, R¹ is C₃₋₆cycloalkyl optionally substituted with one, two, or three groups selected from R^{15a}. In some embodiments, R¹ is C₃₋₆cycloalkyl substituted with one, two, or three groups selected from C₁₋₆alkyl, —OR¹⁰, and —N(R¹⁰)(R¹¹). In some embodiments, R¹ is C₃₋₆cycloalkyl substituted with one group selected from —OR¹⁰ and —N(R¹⁰)(R¹¹) R¹⁰ and R¹¹ are independently selected from hydrogen and C₁₋₆alkyl. In some embodiments, R¹ is unsubstituted C₃₋₆cycloalkyl. In some embodiments, R¹ is unsubstituted cyclopropyl. In some embodiments, R¹ is unsubstituted cyclobutyl.

In some embodiments of a compound of Formula (I) or (II), or a pharmaceutically acceptable salt thereof, R¹ is C₁₋₆haloalkyl.

In some embodiments of a compound of Formula (I) or (II), or a pharmaceutically acceptable salt thereof, R¹ is C₂₋₉heterocycloalkyl optionally substituted with one, two, or three groups selected from R^{15a}. In some embodiments, R¹ is C₂₋₉heterocycloalkyl optionally substituted with one, two, or three groups selected from C₁₋₆alkyl, —OR¹⁰, and —N(R¹⁰)(R¹¹). In some embodiments, R¹ is unsubstituted C₂₋₉heterocycloalkyl.

In some embodiments of a compound of Formula (I) or (II), or a pharmaceutically acceptable salt thereof, R¹ is C₁₋₉heteroaryl optionally substituted with one, two, or three groups selected from R^{15a}.

In some embodiments, R¹ is C₁₋₉heteroaryl optionally substituted with one, two, or three groups selected from C₁₋₆alkyl, —OR¹⁰, and —N(R¹⁰)(R¹¹). In some embodiments, R¹ is unsubstituted C₁₋₉heteroaryl.

In some embodiments, the compound of Formula (I) has a structure of Formula (Ia) or Formula (Ib):

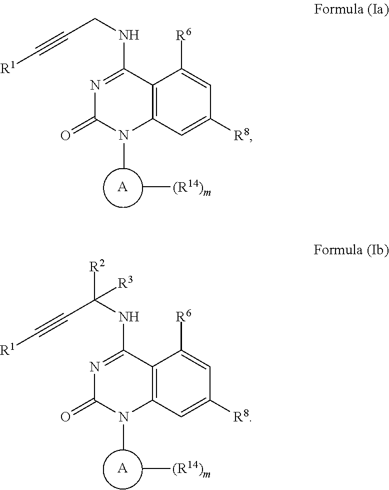

Any combination of the groups described above for the various variables is contemplated herein. Throughout the specification, groups and substituents thereof are chosen by one skilled in the field to provide stable moieties and compounds.

In some embodiments is an MAT2A inhibitor, or a pharmaceutically acceptable salt thereof, selected from a compound found in table 1.

TABLE 1

| Example # | Structure |
|---|---|
| 1 | 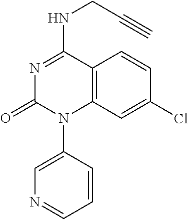 [SMILES: C#CCNc1nc(=O)n(-c2cccnc2)c2cc(Cl)ccc12] |
| 2 | 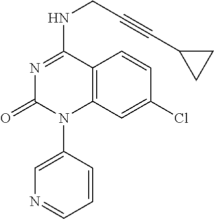 [SMILES: O=c1nc(NCC#CC2CC2)c2ccc(Cl)cc2n1-c1cccnc1] |
| 3 | 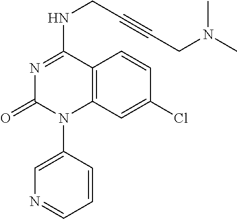 [SMILES: CN(C)CC#CCNc1nc(=O)n(-c2cccnc2)c2cc(Cl)ccc12] |
| 4 | 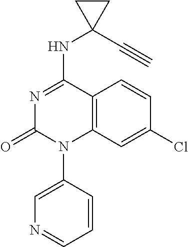 [SMILES: C#CC1(Nc2nc(=O)n(-c3cccnc3)c3cc(Cl)ccc23)CC1] |
| 5 | 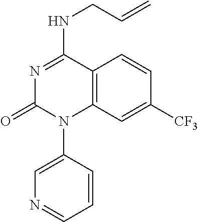 [SMILES: C=CCNc1nc(=O)n(-c2cccnc2)c2cc(C(F)(F)F)ccc12] |
| 6 | 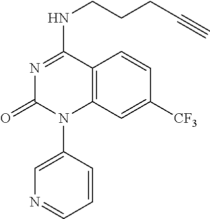 [SMILES: C#CCCCNc1nc(=O)n(-c2cccnc2)c2cc(C(F)(F)F)ccc12] |
| 7 | 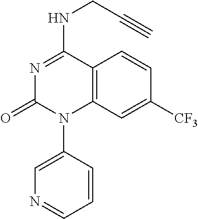 [SMILES: C#CCNc1nc(=O)n(-c2cccnc2)c2cc(C(F)(F)F)ccc12] |
| 8 | 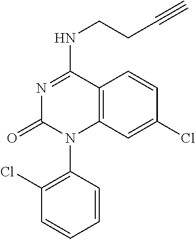 [SMILES: C#CCCNc1nc(=O)n(-c2ccccc2Cl)c2cc(Cl)ccc12] |
| 9 | 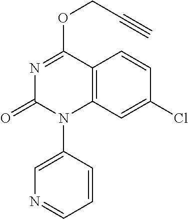 [SMILES: C#CCOc1nc(=O)n(-c2cccnc2)c2cc(Cl)ccc12] |
| 10 | 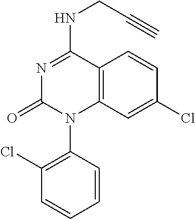 [SMILES: C#CCNc1nc(=O)n(-c2ccccc2Cl)c2cc(Cl)ccc12] |
| 11 | 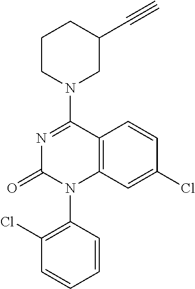 [SMILES: C#CC1CCCN(c2nc(=O)n(-c3ccccc3Cl)c3cc(Cl)ccc23)C1] |
| 12 | 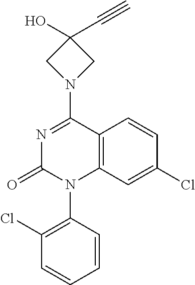 [SMILES: C#CC1(O)CN(c2nc(=O)n(-c3ccccc3Cl)c3cc(Cl)ccc23)C1] |
| 13 | 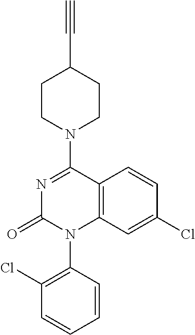 [SMILES: C#CC1CCN(c2nc(=O)n(-c3ccccc3Cl)c3cc(Cl)ccc23)CC1] |
| 14 | 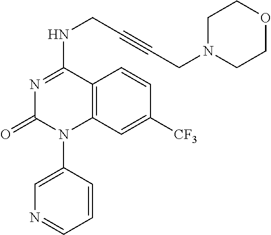 [SMILES: O=c1nc(NCC#CCN2CCOCC2)c2ccc(C(F)(F)F)cc2n1-c1cccnc1] |
| 15 | 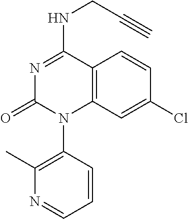 [SMILES: C#CCNc1nc(=O)n(-c2cccnc2C)c2cc(Cl)ccc12] |
| 16 | 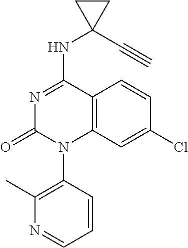 [SMILES: C#CC1(Nc2nc(=O)n(-c3cccnc3C)c3cc(Cl)ccc23)CC1] |
| 17 | 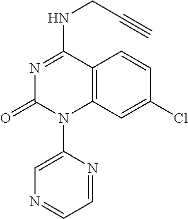 [SMILES: C#CCNc1nc(=O)n(-c2cnccn2)c2cc(Cl)ccc12] |
| 18 | 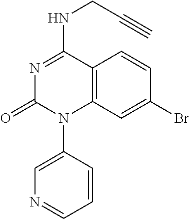 [SMILES: C#CCNc1nc(=O)n(-c2cccnc2)c2cc(Br)ccc12] |
| 19 | 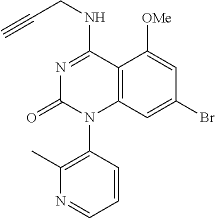 [SMILES: C#CCNc1nc(=O)n(-c2cccnc2C)c2cc(Br)cc(OC)c12] |
| 20 | 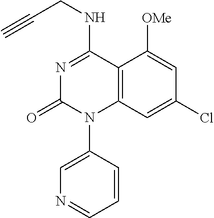 [SMILES: C#CCNc1nc(=O)n(-c2cccnc2)c2cc(Cl)cc(OC)c12] |
| 21 | 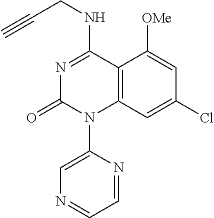 [SMILES: C#CCNc1nc(=O)n(-c2cnccn2)c2cc(Cl)cc(OC)c12] |
| 22 | 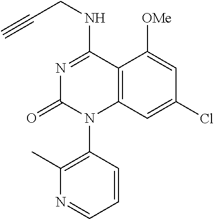 [SMILES: C#CCNc1nc(=O)n(-c2cccnc2C)c2cc(Cl)cc(OC)c12] |
| 23 | 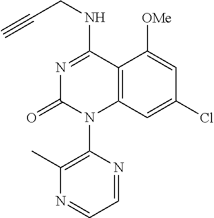 [SMILES: C#CCNc1nc(=O)n(-c2nccnc2C)c2cc(Cl)cc(OC)c12] |
| 24 | 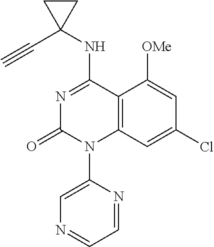 [SMILES: C#CC1(Nc2nc(=O)n(-c3cnccn3)c3cc(Cl)cc(OC)c23)CC1] |
| 25 | 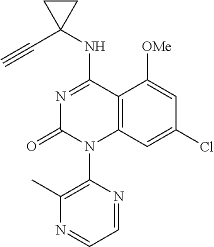 [SMILES: C#CC1(Nc2nc(=O)n(-c3nccnc3C)c3cc(Cl)cc(OC)c23)CC1] |
| 26 | 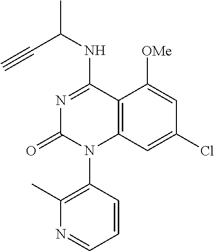 [SMILES: C#CC(C)Nc1nc(=O)n(-c2cccnc2C)c2cc(Cl)cc(OC)c12] |
| 27 | 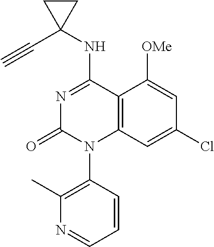 [SMILES: C#CC1(Nc2nc(=O)n(-c3cccnc3C)c3cc(Cl)cc(OC)c23)CC1] |
| 28 | 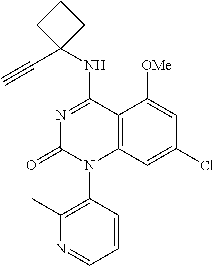 [SMILES: C#CC1(Nc2nc(=O)n(-c3cccnc3C)c3cc(Cl)cc(OC)c23)CCC1] |
| 29 | 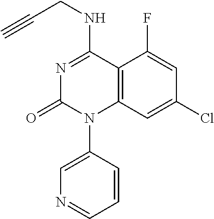 [SMILES: C#CCNc1nc(=O)n(-c2cccnc2)c2cc(Cl)cc(F)c12] |
| 30 | 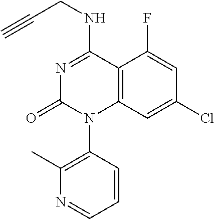 [SMILES: C#CCNc1nc(=O)n(-c2cccnc2C)c2cc(Cl)cc(F)c12] |
| 31 | 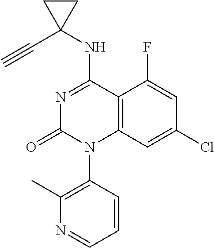 [SMILES: C#CC1(Nc2nc(=O)n(-c3cccnc3C)c3cc(Cl)cc(F)c23)CC1] |
| 32 | 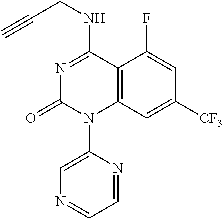 [SMILES: C#CCNc1nc(=O)n(-c2cnccn2)c2cc(C(F)(F)F)cc(F)c12] |
| 33 | 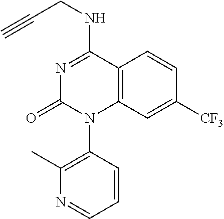 [SMILES: C#CCNc1nc(=O)n(-c2cccnc2C)c2cc(C(F)(F)F)ccc12] |
| 34 | 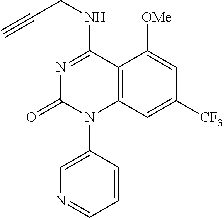 [SMILES: C#CCNc1nc(=O)n(-c2cccnc2)c2cc(C(F)(F)F)cc(OC)c12] |
| 35 | 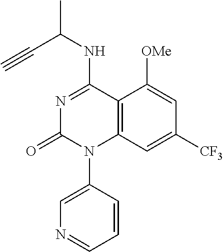 [SMILES: C#CC(C)Nc1nc(=O)n(-c2cccnc2)c2cc(C(F)(F)F)cc(OC)c12] |
| 36 (Compound 1) | 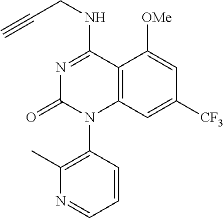 [SMILES: C#CCNc1nc(=O)n(-c2cccnc2C)c2cc(C(F)(F)F)cc(OC)c12] |
| 37 | 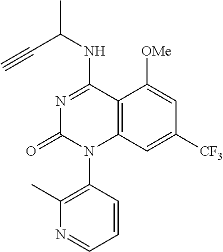 [SMILES: C#CC(C)Nc1nc(=O)n(-c2cccnc2C)c2cc(C(F)(F)F)cc(OC)c12] |
| 38 | 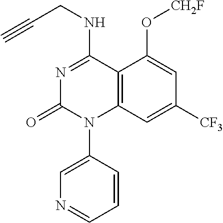 |
| 39 | 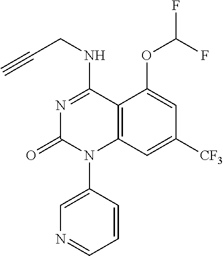 [SMILES: C#CCNc1nc(=O)n(-c2cccnc2)c2cc(C(F)(F)F)cc(OC(F)F)c12] |
| 40 | 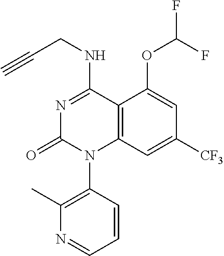 [SMILES: C#CCNc1nc(=O)n(-c2cccnc2C)c2cc(C(F)(F)F)cc(OC(F)F)c12] |
| 41 | 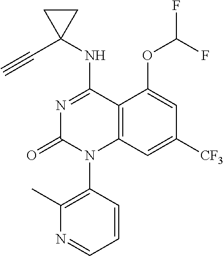 [SMILES: C#CC1(Nc2nc(=O)n(-c3cccnc3C)c3cc(C(F)(F)F)cc(OC(F)F)c23)CC1] |
| 42 | 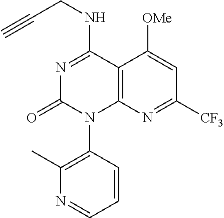 [SMILES: C#CCNc1nc(=O)n(-c2cccnc2C)c2nc(C(F)(F)F)cc(OC)c12] |
| 43 | 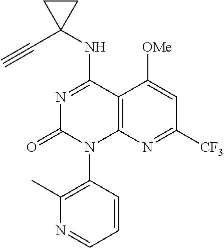 [SMILES: C#CC1(Nc2nc(=O)n(-c3cccnc3C)c3nc(C(F)(F)F)cc(OC)c23)CC1] |
| 44 | 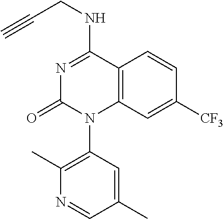 [SMILES: C#CCNc1nc(=O)n(-c2cc(C)cnc2C)c2cc(C(F)(F)F)ccc12] |
| 45 | 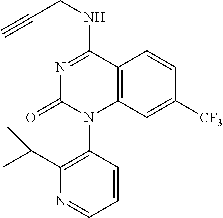 [SMILES: C#CCNc1nc(=O)n(-c2cccnc2C(C)C)c2cc(C(F)(F)F)ccc12] |
| 46 | 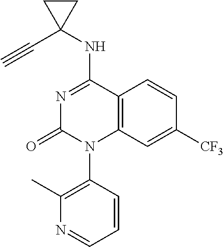 [SMILES: C#CC1(Nc2nc(=O)n(-c3cccnc3C)c3cc(C(F)(F)F)ccc23)CC1] |
| 47 | 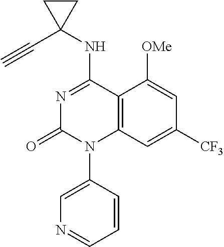 [SMILES: C#CC1(Nc2nc(=O)n(-c3cccnc3)c3cc(C(F)(F)F)cc(OC)c23)CC1] |
| 48 | 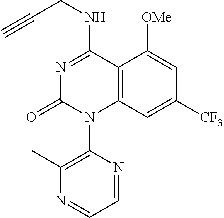 [SMILES: C#CCNc1nc(=O)n(-c2nccnc2C)c2cc(C(F)(F)F)cc(OC)c12] |
| 49 | 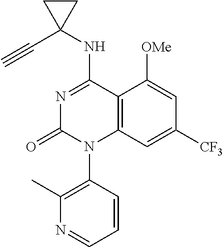 [SMILES: C#CC1(Nc2nc(=O)n(-c3cccnc3C)c3cc(C(F)(F)F)cc(OC)c23)CC1] |
| 50 | 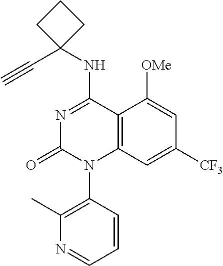 [SMILES: C#CC1(Nc2nc(=O)n(-c3cccnc3C)c3cc(C(F)(F)F)cc(OC)c23)CCC1] |
| 51 | 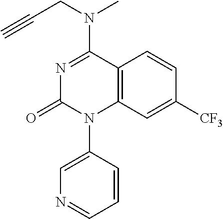 [SMILES: C#CCN(C)c1nc(=O)n(-c2cccnc2)c2cc(C(F)(F)F)ccc12] |
| 52 | 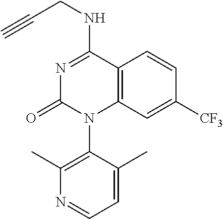 [SMILES: C#CCNc1nc(=O)n(-c2c(C)ccnc2C)c2cc(C(F)(F)F)ccc12] |
| 53 | 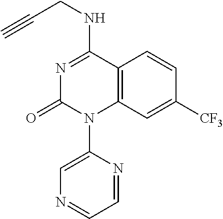 [SMILES: C#CCNc1nc(=O)n(-c2cnccn2)c2cc(C(F)(F)F)ccc12] |
| 54 | 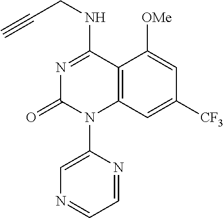 [SMILES: C#CCNc1nc(=O)n(-c2cnccn2)c2cc(C(F)(F)F)cc(OC)c12] |
| 55 | 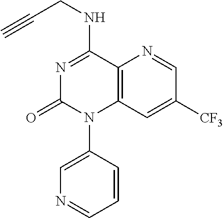 [SMILES: C#CCNc1nc(=O)n(-c2cccnc2)c2cc(C(F)(F)F)cnc12] |
| 56 | 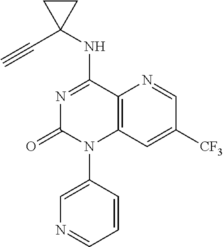 [SMILES: C#CC1(Nc2nc(=O)n(-c3cccnc3)c3cc(C(F)(F)F)cnc23)CC1] |
| 57 | 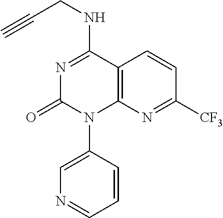 [SMILES: C#CCNc1nc(=O)n(-c2cccnc2)c2nc(C(F)(F)F)ccc12] |
| 58 | 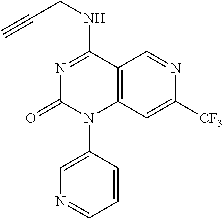 [SMILES: C#CCNc1nc(=O)n(-c2cccnc2)c2cc(C(F)(F)F)ncc12] |
| 59 | 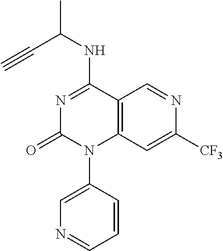 [SMILES: C#CC(C)Nc1nc(=O)n(-c2cccnc2)c2cc(C(F)(F)F)ncc12] |
| 60 | 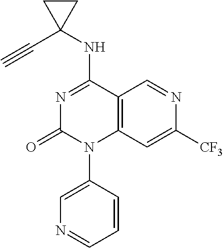 [SMILES: C#CC1(Nc2nc(=O)n(-c3cccnc3)c3cc(C(F)(F)F)ncc23)CC1] |
| 61 | 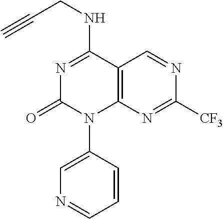 [SMILES: C#CCNc1nc(=O)n(-c2cccnc2)c2nc(C(F)(F)F)ncc12] |
| 62 | 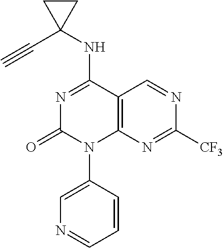 [SMILES: C#CC1(Nc2nc(=O)n(-c3cccnc3)c3nc(C(F)(F)F)ncc23)CC1] |
| 63 | 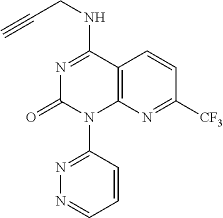 [SMILES: C#CCNc1nc(=O)n(-c2cccnn2)c2nc(C(F)(F)F)ccc12] |
| 64 | 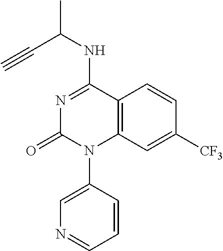 [SMILES: C#CC(C)Nc1nc(=O)n(-c2cccnc2)c2cc(C(F)(F)F)ccc12] |
| 65 | 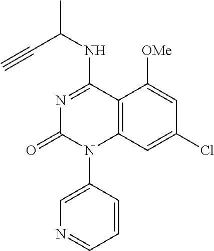 [SMILES: C#CC(C)Nc1nc(=O)n(-c2cccnc2)c2cc(Cl)cc(OC)c12] |
| 66 | 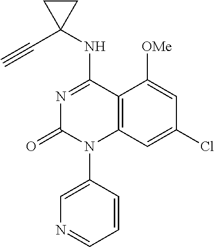 [SMILES: C#CC1(Nc2nc(=O)n(-c3cccnc3)c3cc(Cl)cc(OC)c23)CC1] |
| 67 | 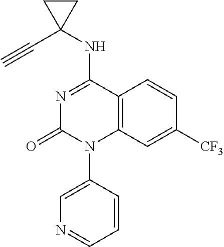 [SMILES: C#CC1(Nc2nc(=O)n(-c3cccnc3)c3cc(C(F)(F)F)ccc23)CC1] |
| 68 | 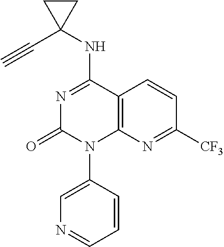 [SMILES: C#CC1(Nc2nc(=O)n(-c3cccnc3)c3nc(C(F)(F)F)ccc23)CC1] |
| 69 | 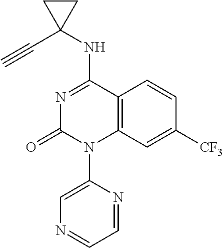 [SMILES: C#CC1(Nc2nc(=O)n(-c3cnccn3)c3cc(C(F)(F)F)ccc23)CC1] |
| 70 | 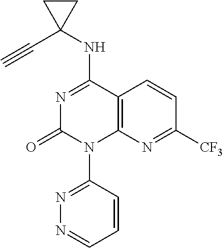 [SMILES: C#CC1(Nc2nc(=O)n(-c3cccnn3)c3nc(C(F)(F)F)ccc23)CC1] |
| 71 | 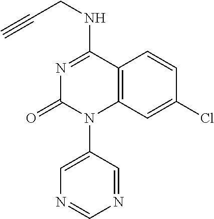 [SMILES: C#CCNc1nc(=O)n(-c2cncnc2)c2cc(Cl)ccc12] |
| 72 | 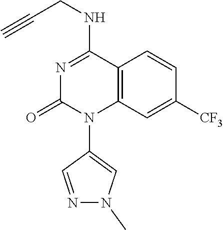 [SMILES: C#CCNc1nc(=O)n(-c2cnn(C)c2)c2cc(C(F)(F)F)ccc12] |
| 73 | 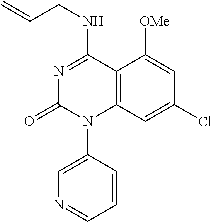 [SMILES: C=CCNc1nc(=O)n(-c2cccnc2)c2cc(Cl)cc(OC)c12] |
| 74 | 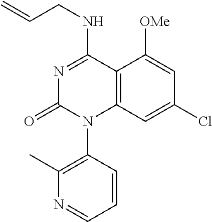 [SMILES: C=CCNc1nc(=O)n(-c2cccnc2C)c2cc(Cl)cc(OC)c12] |
| 75 | 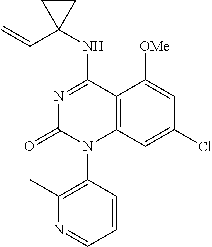 [SMILES: C=CC1(Nc2nc(=O)n(-c3cccnc3C)c3cc(Cl)cc(OC)c23)CC1] |
| 76 | 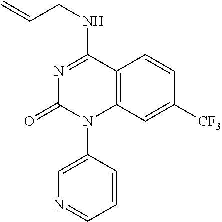 [SMILES: C=CCNc1nc(=O)n(-c2cccnc2)c2cc(C(F)(F)F)ccc12] |
| 77 | 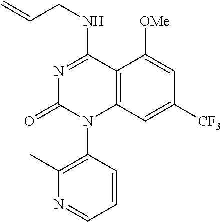 [SMILES: C=CCNc1nc(=O)n(-c2cccnc2C)c2cc(C(F)(F)F)cc(OC)c12] |
| 78 | 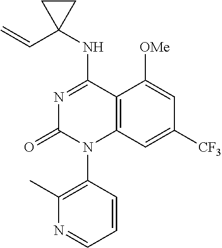 [SMILES: C=CC1(Nc2nc(=O)n(-c3cccnc3C)c3cc(C(F)(F)F)cc(OC)c23)CC1] |
| 79 | 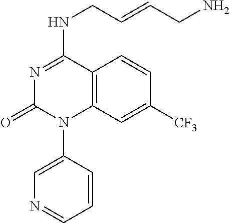 [SMILES: NC/C=C/CNc1nc(=O)n(-c2cccnc2)c2cc(C(F)(F)F)ccc12] |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility.

In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the 1H atoms replaced with ²H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid.

In some embodiments, other acids, such as oxalic, while not in themselves pharmaceutically acceptable, are employed in the preparation of salts useful as intermediates in obtaining the compounds disclosed herein, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Compound 1

In some embodiments disclosed herein, the MAT2A inhibitor is Compound 1. In some embodiments disclosed herein, the MAT2A inhibitor is Compound 1 or a salt thereof.

Compound 1 is 5-methoxy-1-(2-methylpyridin-3-yl)-4-(prop-2-yn-1-ylamino)-7-(trifluoromethyl)quinazolin-2(1H)-one:

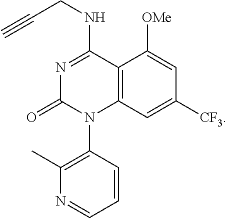 [SMILES: C#CCNc1nc(=O)n(-c2cccnc2C)c2cc(C(F)(F)F)cc(OC)c12]

Compound 1 is a methionine adenosyltransferase II alpha (MAT2A) inhibitor. In some embodiments Compound 1 is in the form of a freebase. In some embodiments Compound 1 is in the form of a pharmaceutically acceptable salt thereof.

In some embodiments, Compound 1 is in the form of an HCl salt. In some embodiments, Compound 1 is in the form of a sulfate salt. In some embodiments, Compound 1 is in the form of a maleate salt. In some embodiments, Compound 1 is in the form of a phosphate salt. In some embodiments, Compound 1 is in the form of a citrate salt. In some embodiments, Compound 1 is in the form of an L-malate salt. In some embodiments, Compound 1 is in the form of a succinate salt. In some embodiments, Compound 1 is in the form of a tosylate salt. In some embodiments, Compound 1 is in the form of a mesylate salt. In some embodiments, Compound 1 is in the form of a besylate salt. In some embodiments, Compound 1 is in the form of an oxalate salt. In some embodiments, Compound 1 is in the form of an esylate salt.

In some embodiments disclosed herein, the MAT2A inhibitor is known in the art and suitable for use in the methods disclosed herein. In some embodiments, the MAT2A inhibitor is selected from a compound disclosed in WO2019191470, WO2020123395, WO2020139992, WO2020243376, WO2021252678, WO2021252679, WO2021252680, WO2021252681, WO2020139991, WO2021139775, WO2021254529, WO2021254529, WO2022053022, WO2022063128, WO2022078403, WO2022052924, WO2022206730, WO2022222911, WO2022228515, WO2022253242, WO2022268180, WO2023066283, WO2023083210, WO2023116390, WO2023116696, WO2023143356, WO2023169554, WO2023185811, WO2023196985, WO2024002024, and WO2024012507, the entire contents of which are hereby incorporated by reference in their entirety. In some embodiments, the MAT2A inhibitor is AG-270 or IDE397, or a pharmaceutically acceptable salt thereof.

### Methods/Combinations

Disclosed herein is a method of treating cancer in a subject in need thereof, the method comprising administering to the subject:
(a) an MAT2A inhibitor or a pharmaceutically acceptable salt thereof; and
(b) an additional agent,
   wherein the combined amount of the MAT2A inhibitor, or a pharmaceutically acceptable salt thereof and the additional agent is therapeutically effective for treating the cancer.

Disclosed herein is a method of treating cancer in a subject in need thereof, the method comprising administering to the subject:
(a) a compound of Formula (I) or Formula (II), or a pharmaceutically acceptable salt thereof:

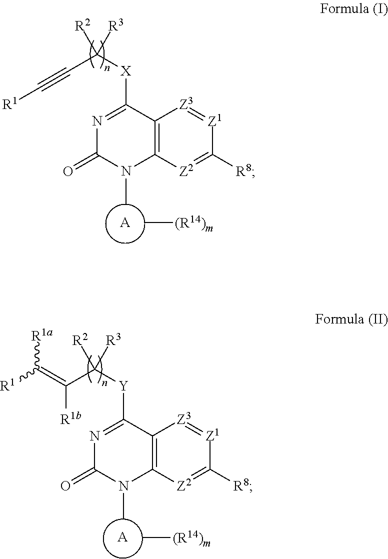

and
(b) an additional agent,
   wherein the combined amount of the compound of Formula (I) or Formula (II), or a pharmaceutically acceptable salt thereof and the additional agent is therapeutically effective for treating the cancer. In some embodiments, the additional agent is an anti-cancer agent.

In some embodiments, the compound is a compound of Formula (I). In some embodiments, the compound is a salt of a compound of Formula (I).

In some embodiments, the compound is a compound of Formula (II). In some embodiments, the compound is a salt of a compound of Formula (II).

Disclosed herein is a method of treating cancer in a subject in need thereof, the method comprising administering to the subject:
(a) 5-methoxy-1-(2-methylpyridin-3-yl)-4-(prop-2-yn-1-ylamino)-7-(trifluoromethyl)quinazolin-2(1H)-one:

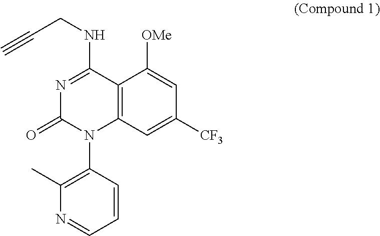 [SMILES: C#CCNc1nc(=O)n(-c2cccnc2C)c2cc(C(F)(F)F)cc(OC)c12]

or a pharmaceutically acceptable salt thereof; and
(b) an additional agent.

In some embodiments, Compound 1 or the pharmaceutically acceptable salt thereof and the additional agent are administered in a therapeutically effective amount for treating the cancer. In some embodiments, the combined amount of Compound 1 or the pharmaceutically acceptable salt thereof and the additional agent is therapeutically effective for treating the cancer.

In some embodiments, the cancer is a gastric cancer, primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the cancer is liver cancer, colon cancer, pancreatic cancer, prostate cancer, lung cancer, breast cancer, or T cell leukemia.

In some embodiments, the cancer is liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.

In some embodiments, the cancer is a MTAP-deficient cancer. In some embodiments, the MTAP-deficient cancer is a primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In other embodiments, the MTAP-deficient cancer is MTAP-deficient lung cancer, MTAP-deficient pancreatic cancer, MTAP-deficient esophageal cancer, MTAP-deficient colorectal cancer, MTAP-deficient kidney cancer, or MTAP-deficient leukemia, such as acute myeloid leukemia (AML).

In some embodiments, the MTAP-deficient cancer is MTAP-deficient lung cancer, such as NSCLC.

In other embodiments, the MTAP-deficient cancer is MTAP-deficient pancreatic cancer, such as PDAC. In some embodiments, the MTAP-deficient cancer is MTAP-deficient esophageal cancer.

In some embodiments, the MTAP-deficient cancer is MTAP-deficient colorectal cancer.

In some embodiments, the MTAP-deficient cancer is MTAP-deficient kidney cancer.

In some embodiments, the MTAP-deficient cancer is MTAP-deficient leukemia, such as acute myeloid leukemia (AML).

In some embodiments, the cancer is a MTAP wild type cancer. In some embodiments, the MTAP wild type cancer is a primary leukemia, hematological malignancy, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the MTAP wild type cancer is MTAP wild type lung cancer, MTAP wild type pancreatic cancer, MTAP wild type esophageal cancer, MTAP wild type colorectal cancer, MTAP wild type kidney cancer, or MTAP wild type leukemia, such as acute myeloid leukemia (AML).

In some embodiments, the MTAP wild type cancer is MTAP wild type lung cancer, such as NSCLC.

In some embodiments, the MTAP wild type cancer is MTAP wild type pancreatic cancer, such as PDAC.

In some embodiments, the MTAP wild type cancer is MTAP wild type esophageal cancer.

In some embodiments, the MTAP wild type cancer is MTAP wild type colorectal cancer.

In some embodiments, the MTAP wild type cancer is MTAP wild type kidney cancer.

In some embodiments, the MTAP wild type cancer is MTAP wild type leukemia, such as acute myeloid leukemia (AML).

In some embodiments, the cancer is a cancer that responds to a reduction in SAM as a result of administering an MAT2A inhibitor. In some embodiments, the cancer that responds to a reduction in SAM as a result of administering an MAT2A inhibitor is a primary leukemia, hematological malignancy, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the cancer that responds to a reduction in SAM as a result of administering an MAT2A inhibitor is lung cancer, pancreatic cancer, esophageal cancer, colorectal cancer, kidney cancer, or leukemia, such as acute myeloid leukemia (AML).

In some embodiments, the cancer that responds to a reduction in SAM as a result of administering an MAT2A inhibitor is lung cancer, such as NSCLC.

In some embodiments, the cancer that responds to a reduction in SAM as a result of administering an MAT2A inhibitor is pancreatic cancer, such as PDAC.

In some embodiments, the cancer that responds to a reduction in SAM as a result of administering an MAT2A inhibitor is esophageal cancer.

In some embodiments, the cancer that responds to a reduction in SAM as a result of administering an MAT2A inhibitor is colorectal cancer.

In some embodiments, the cancer that responds to a reduction in SAM as a result of administering an MAT2A inhibitor is kidney cancer.

In some embodiments, the cancer that responds to a reduction in SAM as a result of administering an MAT2A inhibitor is leukemia, such as acute myeloid leukemia (AML).

In some embodiments of a method of treating cancer, the additional agent is a PARP inhibitor, a CHK1 inhibitor, a MDM2 inhibitor, a hypomethylating agent, an mTOR inhibitor, an ATM inhibitor, a CDK 4/6 inhibitor, a BCL-2 inhibitor, a PRMT5 inhibitor, a PRMT1 inhibitor, an ATR inhibitor, a WEE 1 inhibitor, an APE 1 inhibitor, a topoisomerase inhibitor, a taxane, an immune checkpoint inhibitor, a CDK7 inhibitor, a CDK9 inhibitor, a DNA synthesis inhibitor, an antimetabolite, an AURORA inhibitor, a microtubule stabilizer, a DNA cross-linker, a vinca alkaloid, an alkylating agent, a PRMT6 inhibitor, a PRMT7 inhibitor, a PRMT9 inhibitor, a KRAS inhibitor, an EGFR inhibitor, a VEGFR inhibitor, an aromatase inhibitor, a mitotic inhibitor, a radiopharmaceutical agent, a cytotoxic agent, or any combination thereof.

In some embodiments of a method of treating cancer, the additional agent is a PARP inhibitor.

In some embodiments of a method of treating cancer, the PARP inhibitor is olaparib (AZD2281), veliparib (ABT-888), rucaparib, talazoparib (BMN 673), AG-14361, INO-1001 (3-aminobenzamide), A-966492, PJ34 HCl, niraparib, UPF 1069, ME0328, RK-287107, pamiparib (BGB-290), NMS-P118, E7449, picolinamide, benzamide, NU1025, iniparib (B SI-201), AZD2461, BGP-15 2HC1, XAV-939, 4-hydroxyquinazoline, NVP-TNKS656, MN 64, or G007-LK, or a pharmaceutically acceptable salt thereof.

In some embodiments of a method of treating cancer, the PARP inhibitor is olaparib (AZD2281), rucaparib, talazoparib (BMN 673), niraparib, or talazoparib (BMN 673), or a pharmaceutically acceptable salt thereof.

In some embodiments of a method of treating cancer, the PARP inhibitor is olaparib (AZD2281) or a pharmaceutically acceptable salt thereof.

In some embodiments of a method of treating cancer, the PARP inhibitor is talazoparib (BMN 673) or a pharmaceutically acceptable salt thereof.

In some embodiments of a method of treating cancer, the additional agent is a CHK1 inhibitor.

In some embodiments of a method of treating cancer, the CHK1 inhibitor is AZD7762, rabusertib (LY2603618), MK-8776 (SCH 900776), CHIR-124, PF-477736, VX-803 (M4344), GDC-0575 (ARRY-575), SAR-020106, CCT245737, PD0166285, or prexasertib (LY2606368), or a pharmaceutically acceptable salt thereof.

In some embodiments of a method of treating cancer, the CHK1 inhibitor is AZD7762 or Rabusertib (LY2603618), or a pharmaceutically acceptable salt thereof.

In some embodiments of a method of treating cancer, the CHK1 inhibitor is AZD7762 or a pharmaceutically acceptable salt thereof.

In some embodiments of a method of treating cancer, the CHK1 inhibitor is Rabusertib (LY2603618) or a pharmaceutically acceptable salt thereof.

In some embodiments of a method of treating cancer, the additional agent is a MDM2 inhibitor.

In some embodiments of a method of treating cancer, the MDM2 inhibitor is nutlin-3, NSC 207895, nutlin-3a, nutlin-3b, MX69, NVP-CGM097, MI-773 (SAR405838), idasanutlin (RG-7388), RG-7112, HDM201 (Siremadlin), YH239-EE, (−)-parthenolide, or serdemetan (JNJ-26854165) or a pharmaceutically acceptable salt thereof.

In some embodiments of a method of treating cancer, the MDM2 inhibitor is nutlin-3 or Serdemetan (JNJ-26854165) or a pharmaceutically acceptable salt thereof.

In some embodiments of a method of treating cancer, the MDM2 inhibitor is nutlin-3 or a pharmaceutically acceptable salt thereof.

In some embodiments of a method of treating cancer, the MDM2 inhibitor is serdemetan (JNJ-26854165) or a pharmaceutically acceptable salt thereof.

In some embodiments of a method of treating cancer, the additional agent is a hypomethylating agent.

In some embodiments of a method of treating cancer, the hypomethylating agent is decitabine, azacitidine (5-azacytidine), RG108, thioguanine, zebularine, SGI-1027, CM272, 2′-deoxy-5-fluorocytidine, procainamide, bobcat339, gamma-oryzanol, thujaplicin, or (−)-epigallocatechin gallate, or a pharmaceutically acceptable salt thereof.

In some embodiments of a method of treating cancer, the hypomethylating agent is decitabine or azacitidine (5-Azacytidine), or a pharmaceutically acceptable salt thereof.

In some embodiments of a method of treating cancer, the hypomethylating agent is procainamide or a pharmaceutically acceptable salt thereof.

In some embodiments of a method of treating cancer, the hypomethylating agent is decitabine or a pharmaceutically acceptable salt thereof.

In some embodiments of a method of treating cancer, the hypomethylating agent is azacitidine (5-azacytidine) or a pharmaceutically acceptable salt thereof.

In some embodiments of a method of treating cancer, the additional agent is a mTOR inhibitor.

In some embodiments of a method of treating cancer, the mTOR inhibitor is dactolisib (BEZ235), rapamycin (sirolimus), everolimus (RAD001), AZD8055, temsirolimus (CCI-779), PI-103, KU-0063794, torkinib (PP242), ridaforolimus (deforolimus, MK-8669), sapanisertib (MLN0128), voxtalisib (XL765), torin 1, torin 2, omipalisib (GSK2126458), OSI-027, PF-04691502, apitolisib (GDC-0980), GSK1059615, gedatolisib (PKI-587), WYE-354, vistusertib (AZD2014), WYE-125132 (WYE-132), PP121, WYE-687, WAY-600, ETP-46464, GDC-0349, XL388, GNE-477, bimiralisib (PQR309), SF2523, CZ415, paxalisib (GDC-0084), CC-115, onatasertib (CC 223), voxtalisib (XL765), zotarolimus (ABT-578), Tacrolimus (FK506), BGT226 maleate (NVP-BGT226 maleate), palomid 529 (P529), LY3023414 (samotolisib), biolimus-7, biolimus-9, azathioprine, campath 1H, or chrysophanic acid, or a pharmaceutically acceptable salt thereof.

In some embodiments of a method of treating cancer, the mTOR inhibitor is everolimus (RAD001) or a pharmaceutically acceptable salt thereof.

In some embodiments of a method of treating cancer, the additional agent is an ATM inhibitor.

In some embodiments of a method of treating cancer, the ATM inhibitor is KU-55933, KU-60019, wortmannin, torin 2, CP-466722, ETP-46464, CGK 733, AZ32, AZD1390, AZ31, or AZD0156, or a pharmaceutically acceptable salt thereof.

In some embodiments of a method of treating cancer, the ATM inhibitor is KU-60019 or a pharmaceutically acceptable salt thereof.

In some embodiments of a method of treating cancer, the additional agent is a CDK 4/6 inhibitor.

In some embodiments of a method of treating cancer, the CDK 4/6 inhibitor is palbociclib (PD-0332991), alvocidib, AT7519, JNJ-7706621, PHA-793887, BMS-265246, milciclib (PHA-848125), R547, riviciclib (P276-00), MC180295, G1T38, abemaciclib, ON123300, AT7519, purvalanol A, SU9516, ribociclib (LEE011), or BSJ-03-123, or a pharmaceutically acceptable salt thereof.

In some embodiments of a method of treating cancer, the CDK 4/6 inhibitor is palbociclib (PD-0332991) or a pharmaceutically acceptable salt thereof.

In some embodiments of a method of treating cancer, the additional agent is a BCL-2 inhibitor.

In some embodiments of a method of treating cancer, the BCL-2 inhibitor is ABT-737, navitoclax (ABT-263), obatoclax (GX15-070), TW-37, venetoclax (ABT-199), AT101, HA14-1, sabutoclax, S55746, or gambogic acid, or a pharmaceutically acceptable salt thereof.

In some embodiments of a method of treating cancer, the BCL-2 inhibitor is venetoclax (ABT-199) or a pharmaceutically acceptable salt thereof.

In some embodiments of a method of treating cancer, the additional agent is a type I PRMT inhibitor.

In some embodiments of a method of treating cancer, the type I PRMT inhibitor is selected from a compound disclosed in WO2014153226, WO2021023609, or WO2022256808, the entire contents of which are hereby incorporated by reference in their entirety.

In some embodiments of a method of treating cancer, the type I PRMT inhibitor is

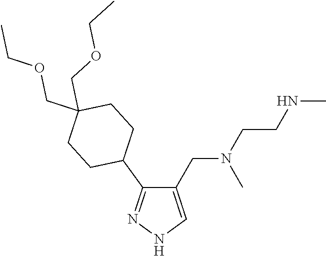 [SMILES: CCOCC1(COCC)CCC(c2n[nH]cc2CN(C)CCNC)CC1]

or a pharmaceutically acceptable salt thereof.

In some embodiments of a method of treating cancer, the Type I PRMT inhibitor is a protein arginine methyltransferase 1 (PRMT1) inhibitor or a protein arginine methyltransferase 6 (PRMT6) inhibitor.

In some embodiments of a method of treating cancer, the additional agent is a PRMT1 inhibitor.

In some embodiments of a method of treating cancer, the PRMT1 inhibitor is GSK3368715 (EPZ019997), C7280948, EPZ020411 2HC1, MSO₂3, furamidine, C 21, or TC-E 5003, or a pharmaceutically acceptable salt of a listed compound.

In some embodiments of a method of treating cancer, the PRMT1 inhibitor is GSK3368715 (EPZ019997) or a pharmaceutically acceptable salt thereof.

In some embodiments of a method of treating cancer, the additional agent is a PRMT6 inhibitor.

In some embodiments of a method of treating cancer, the PRMT6 inhibitor is SGC 6870 or a pharmaceutically acceptable salt thereof

In some embodiments of a method of treating cancer, the additional agent is a type II PRMT inhibitor.

In some embodiments of a method of treating cancer, the Type II PRMT inhibitor is a protein arginine methyltransferase 5 (PRMT5) inhibitor, a protein arginine methyltransferase 7 (PRMT7) inhibitor, or a protein arginine methyltransferase 9 (PRMT9) inhibitor.

In some embodiments of a method of treating cancer, the additional agent is a PRMT5 inhibitor.

In some embodiments of a method of treating cancer, the PRMT5 inhibitor is JNJ-64619178 (AGI-931), HLCL-61, GSK591, EPZ015666 (GSK3235025), GSK3326595 (EPZ015938; AGI-219), TNG908, TNG462, AMG193, AMG9747, MRTX1719, P305-05313, CTS3157, PH-020-803, or AZ-PRMT5i-1, or a pharmaceutically acceptable salt thereof.

In some embodiments of a method of treating cancer, the PRMT5 inhibitor is TNG908, TNG462, AMG193, AMG9747, MRTX1719, or P305-05313, or a pharmaceutically acceptable salt thereof.

In some embodiments of a method of treating cancer, the PRMT5 inhibitor is GSK3326595 (EPZ015938; AGI-219) or JNJ-64619178 (AGI-931), or a pharmaceutically acceptable salt thereof.

In some embodiments of a method of treating cancer, the PRMT5 inhibitor is GSK3326595 or a pharmaceutically acceptable salt thereof.

In some embodiments of a method of treating cancer, the PRMT5 inhibitor is JNJ-64619178 (AGI-931) or a pharmaceutically acceptable salt thereof.

In some embodiments of a method of treating cancer, the PRMT5 inhibitor is

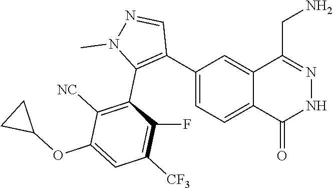

or a pharmaceutically acceptable salt thereof.

In some embodiments of a method of treating cancer, the PRMT5 inhibitor is

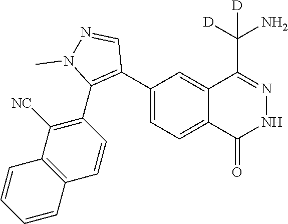 [SMILES: [2H]C([2H])(N)c1n[nH]c(=O)c2ccc(-c3cnn(C)c3-c3ccc4ccccc4c3C#N)cc12]

or a pharmaceutically acceptable salt thereof.

In some embodiments of a method of treating cancer, the PRMT5 inhibitor is

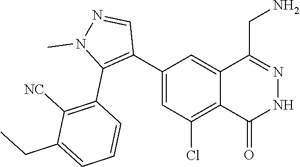 [SMILES: CCc1cccc(-c2c(-c3cc(Cl)c4c(=O)[nH]nc(CN)c4c3)cnn2C)c1C#N]

or a pharmaceutically acceptable salt thereof.

In some embodiments of a method of treating cancer, the PRMT5 inhibitor is selected from a compound disclosed in WO2021050915, WO2021086879, WO2021/163344, WO2022/026892, WO 2022/256806, WO2023036974, WO2021050915, WO2022192745, WO2023278564, WO2022132914, WO2022169948, WO2022115377, WO2021163344, WO2021086879, WO2022026892, U.S. Ser. No. 11/077,101, Malik, R., et al. AACR Annual Meeting, 2021, Abstract Number 1140, or Bonday, Z. Q., et al., ACS Med. Chem. Lett. 2018, 9, 612-617, the entire contents of which are hereby incorporated by reference in their entirety.

In some embodiments of a method of treating cancer, the additional agent is a PRMT7 inhibitor.

In some embodiments of a method of treating cancer, the PRMT7 inhibitor is SGC 3027 or a pharmaceutically acceptable salt thereof

In some embodiments of a method of treating cancer, the additional agent is a PRMT9 inhibitor.

In some embodiments of a method of treating cancer, the additional agent is an ATR inhibitor.

In some embodiments of a method of treating cancer, the ATR inhibitor is RP-3500, M-6620, berzosertib (M-6620, VX-970; VE-822), AZD-6738, AZ-20, M-4344 (VX-803), BAY-1895344, M-1774, IMP-9064, nLs-BG-129, SC-0245, BKT-300, ART-0380, ATRN-119, ATRN-212, or NU-6027, or a pharmaceutically acceptable salt thereof.

In some embodiments of a method of treating cancer, the additional agent is a WEE 1 inhibitor.

In some embodiments of a method of treating cancer, the WEE1 inhibitor is AZD1775 (MK1775), ZN-c3, debio 0123, IMP7068, SDR-7995, SDR-7778, NUV-569, PD0166285, PD0407824, SC-0191, DC-859/A, bosutinib, or Bos-I, or a pharmaceutically acceptable salt thereof.

In some embodiments of a method of treating cancer, the additional agent is a topoisomerase inhibitor.

In some embodiments of a method of treating cancer, the topoisomerase inhibitor is epipodopyyllotoxin, SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, thiotepa, cyclosphosphamide, amsacrine, etoposide, etoposide phosphate, teniposide, daunorubicin, mitoxantrone, amsacrine, ellipticines, aurintricarboxylic acid, doxorubicin, or HU-331, or a pharmaceutically acceptable salt thereof.

In some embodiments of a method of treating cancer, the additional agent is a cytotoxic agent. In some embodiments, the cytotoxic agent is an alkylating agent, a cytotoxic antibiotic agent, an antimetabolite, a vinca alkaloid, a platinum drug, a taxane, or a topoisomerase inhibitor. In some embodiments of a method of treating cancer, the additional agent is a cytotoxic antibiotic agent. In some embodiments, the cytotoxic antibiotic agent is an anthracycline (e.g., doxorubicin and valrubicin). In some embodiments, the cytotoxic antibiotic agent is a non-anthracycline (e.g., bleomycin and dactinomycin). In some embodiments, the additional agent is an agent that is detrimental to the viability of cells.

In some embodiments of a method of treating cancer, the additional agent is platinum drug. In some embodiments of a method of treating cancer, the platinum drug is cisplatin, carboplatin, oxaliplatin, nedaplatin, triplatin tetranitrate, phenanthriplatin, picoplatin, or satraplatin.

In some embodiments of a method of treating cancer, the additional agent is a taxane.

In some embodiments of a method of treating cancer, the taxane is docetaxel, paclitaxel, accatin III, 10-deacetyltaxol, 7-xylosyl-10-deacetyltaxol, chalcomenite, 10-deacetyl-7-epitaxol, 7-epitaxol, 10-deacetylbaccatin III, or 10-deacetyl chalcomenite, or a pharmaceutically acceptable salt thereof.

In some embodiments of a method of treating cancer, the taxane is docetaxel or a pharmaceutically acceptable salt thereof.

In some embodiments of a method of treating cancer, the additional agent is an immune checkpoint inhibitor.

In some embodiments of a method of treating cancer, the immune checkpoint inhibitor is nivolumab, pembrolizumab, pidilizumab, AMP-224, PF-06801591, MEDI0680, PDR001, REGN2810, SHR-12-1, TSR-042, CA-170, atezolizumab, durvalumab, KN035, and BMS-936559, ipilimumab, tremelimumab, AGEN1884, AGEN2041, BMS-986016, GSK2831781, IMP321, LAG525, MGD013, or TSR-022, or a pharmaceutically acceptable salt thereof.

In some embodiments of a method of treating cancer, the immune checkpoint inhibitor is nivolumab, pembrolizumab, pidilizumab, or a pharmaceutically acceptable salt thereof.

In some embodiments of a method of treating cancer, the immune checkpoint inhibitor is nivolumab or a pharmaceutically acceptable salt thereof.

In some embodiments of a method of treating cancer, the immune checkpoint inhibitor is pembrolizumab or a pharmaceutically acceptable salt thereof.

In some embodiments of a method of treating cancer, the immune checkpoint inhibitor is pidilizumab, or a pharmaceutically acceptable salt thereof.

In some embodiments of a method of treating cancer, the additional agent is a CDK7 inhibitor.

In some embodiments of a method of treating cancer, the CDK7 inhibitor is LDC4297, THZ1, THZ2, YKL-5-124, BS-181, samuraciclib, LY3405105, PHA-793887, SNS-032 (BMS-387032), PF-562271, or milciclib (PHA-848125), or a pharmaceutically acceptable salt thereof.

In some embodiments of a method of treating cancer, the additional agent is a CDK9 inhibitor.

In some embodiments of a method of treating cancer, the CDK9 inhibitor is SNS-032 (BMS-387032), LY2857785, alvocidib, or riviciclib hydrochloride (P276-00), or a pharmaceutically acceptable salt thereof.

In some embodiments of a method of treating cancer, the additional agent is a DNA synthesis inhibitor.

In some embodiments of a method of treating cancer, the DNA synthesis inhibitor is 5-fluorouracil (5-FE1), 6-mercaptopurine (6-MP), capecitabine, cytarabine, floxuridine, fludarabine, gemcitabine, hydroxyurea, methotrexate, or pemetrexed, or a pharmaceutically acceptable salt thereof.

In some embodiments of a method of treating cancer, the DNA synthesis inhibitor is pemetrexed or a pharmaceutically acceptable salt thereof.

In some embodiments of a method of treating cancer, the additional agent is an antimetabolite.

In some embodiments of a method of treating cancer, the antimetabolite is 5-fluorouracil (5-FU), 6-mercaptopurine (6-MP), capecitabine, cytarabine, Floxuridine, fludarabine, gemcitabine, hydroxy carbamide, methotrexate, pemetrexed, or phototrexate, or a pharmaceutically acceptable salt thereof.

In some embodiments of a method of treating cancer, the antimetabolite is pemetrexed, 5-fluorouracil (5-FU), or pemetrexed, or a pharmaceutically acceptable salt thereof.

In some embodiments of a method of treating cancer, the antimetabolite is pemetrexed or a pharmaceutically acceptable salt thereof.

In some embodiments of a method of treating cancer, the additional agent is an AURORA inhibitor.

In some embodiments of a method of treating cancer, the AURORA inhibitor is alisertib (MLN8237), tozasertib (VX-680, MK-0457), barasertib (AZDI 152-HQPA), ZM 447439, MLN8054, danusertib (PHA-739358), AT9283, JNJ-7706621, hesperadin, aurora A inhibitor I (TC-S 7010), KW-2449, SNS-314, ENMD-2076, PHA-680632, MK-5108 (VX-689), CYC116, AMG-900, PF-03814735, CCT 129202, GSK1070916, TAK-901, CCT137690, MK-8745, ENMD-2076, aurora kinase inhibitor III, SNS-314 mesylate, BI-847325, reversine, or ABT-348, or a pharmaceutically acceptable salt thereof.

In some embodiments of a method of treating cancer, the additional agent is a KRAS inhibitor.

In some embodiments of a method of treating cancer, the KRAS inhibitor is a KRAS G12C inhibitor.

In some embodiments of a method of treating cancer, the KRAS inhibitor is a KRAS G12D inhibitor.

In some embodiments of a method of treating cancer, the KRAS inhibitor is a KRAS G12S inhibitor.

In some embodiments of a method of treating cancer, the KRAS inhibitor is 6H05, adagrasib, ARS-1323, ARS-1323-alkyne, ARS-1620, ARS-1630, ARS-853, ASP2453, AZD4625, BAY-293, BI-0474, BI-2852, BI-3406, divarasib, G12Si-1, G12Si-5 formic, G12Si-5, garsorasib, K₂O, KRAS G12C inhibitor 1, KRAS G12C inhibitor 2, KRAS G12C inhibitor 3, KRAS G12C inhibitor 4, KRAS G12C inhibitor 5, KRAS G12C inhibitor 13, KRAS G12C inhibitor 14, KRAS G12C inhibitor 15, KRAS G12C inhibitor 16, KRAS G12C inhibitor 17, KRAS G12C inhibitor 18, KRAS G12C inhibitor 23, KRAS G12C inhibitor 24, KRAS G12C inhibitor 25, KRAS G12C inhibitor 26, KRAS G12C inhibitor 27, KRAS G12C inhibitor 28, KRAS G12C inhibitor 32, KRAS G12C inhibitor 43, KRAS G12C inhibitor 44, KRAS G12C inhibitor 45, KRAS G12C inhibitor 46, KRAS G12C inhibitor 47, KRAS G12C inhibitor 48, KRAS G12C inhibitor 49, KRAS G12C inhibitor 50, KRAS G12C inhibitor 51, KRAS G12C inhibitor 52, KRAS G12C inhibitor 53, KRAS G12C inhibitor 54, KRAS G12C inhibitor 55, KRAS G12C inhibitor 57, K-Ras G12C-IN-2, KRAS G12D inhibitor 3, KRAS G12D inhibitor 7, KRAS G12D inhibitor 14, KRAS G12D inhibitor 16, KRAS G12D inhibitor 17, KRAS inhibitor-3, KRAS inhibitor-6, KRAS inhibitor-7, KRAS inhibitor-8, KRAS inhibitor-10, KRAS inhibitor-11, KRAS inhibitor-12, KRAS inhibitor-13, KRAS inhibitor-14, KRAS inhibitor-15, KRAS inhibitor-16, KRAS inhibitor-17, KRAS inhibitor-18, KRAS inhibitor-20, K-Ras(G12C) inhibitor 6, KRpep-2d, LC-2, MRTX1133, MRTX-1257, MRTX849 acid, MRTX-EX185 formic, opnurasib, Pan KRas-IN-1, PROTAC K-Ras Degrader-1, RM-018, SAH-SOS1A, SOS1-IN-4, SOS1-IN-9, sotorasib, or ZG1077, or a pharmaceutically acceptable salt thereof.

In some embodiments of a method of treating cancer, the KRAS inhibitor is ARS-3248 (JNJ-74699157), AMG510, MRTX849, MRTX1133, ASP245, 3GDC6036, BI-2852, BI 1701963, mRNA-5671, JDQ443, RAS(ON) inhibitors, BBP-454, RM-018, RMC1-6291, or RMC1-6236, or a pharmaceutically acceptable salt thereof.

In some embodiments of a method of treating cancer, the KRAS inhibitor is adagrasib, divarasib, garsorasib, opnurasib, or sotorasib, or a pharmaceutically acceptable salt thereof.

In some embodiments of a method of treating cancer, the KRAS inhibitor is sotorasib or a pharmaceutically acceptable salt thereof.

In some embodiments of a method of treating cancer, the KRAS inhibitor is adagrasib or a pharmaceutically acceptable salt thereof.

In some embodiments of a method of treating cancer, the KRAS inhibitor is selected from a compound disclosed in WO2018119183, WO2018217651, WO2019051291, WO2019213526, WO2019213516, WO2019217691, WO2019232419, WO2019241157, WO2020106640, WO2021081212, WO2022083569, WO2022093856, WO2022232332, WO2022232331, WO2020146613, WO2020097537, WO2015054572, WO2020177629, WO2019141250, WO2020081282, WO2020085493, WO2018143315, WO2018206539, WO2019110751, WO2019195609, WO2021207172, WO2021041671, WO2021150613, WO2021142252, WO2021152149, WO2021248090, WO2021216770, WO2022002102, WO2022031678, U.S. Ser. No. 10/662,204B2, U.S. Ser. No. 10/689,377B2, U.S. Ser. No. 10/689,377B2, U.S. Ser. No. 10/689,377B2, U.S. Ser. No. 10/689,377B2, or U.S. Ser. No. 10/519,146B2, the entire contents of which are hereby incorporated by reference in their entirety.

In some embodiments of a method of treating cancer, the additional agent is an EGFR inhibitor.

In some embodiments of a method of treating cancer, the EGFR inhibitor is Erlotinib (OSI-774) HCl, Gefitinib (ZD1839), Lapatinib (GW-572016) Ditosylate, Afatinib (BIBW2992), Saracatinib (AZD0530), Vandetanib (ZD6474), Neratinib (HKI-272), Canertinib (CI-1033), Lapatinib (GW-572016), AG-490 (Tyrphostin B42), CP-724714, Dacomitinib (PF-00299804), WZ4002, Sapitinib (AZD8931), CUDC-101, AG-1478 (Tyrphostin AG-1478), PD153035 HCl, Pelitinib (EKB-569), AEE788 (NVP-AEE788), AC480 (BMS-599626), AP26113-analog (ALK-IN-1), OSI-420, WZ3146, Allitinib tosylate, Rociletinib (CO-1686), Varlitinib, Icotinib (BPI-2009H), TAK-285, WHI-P154, Daphnetin, PD168393, CNX-2006, Tyrphostin 9, AG-18, 0-Demethyl-Gefitinib, AST-1306, BDTX-189, Epertinib hydrochloride, JND3229, BI-4020, Tyrphostin AG-528, AG 556, Canertinib dihydrochloride, EGFR Inhibitor, Gefitinib-based PROTAC 3, SU5214, RG 13022, TQB3804 (EGFR-IN-7), zipalertinib, Pyrotinib (SHR-1258) dimaleate, PD153035, AG 494, AG 555, Theliatinib (HMPL-309), Avitinib (ACO0010), Lazertinib, Gefitinib hydrochloride, Cetuximab (anti-EGFR), Lifirafenib (BGB-283), Nazartinib (EGF816), Brigatinib (AP26113), Tucatinib, Zorifertinib (AZD3759), Afatinib (BIBW2992) Dimaleate, Erlotinib (OSI-774), CL-387785 (EKI-785), Poziotinib (HM781-36B), Osimertinib (AZD9291), AZ5104, AV-412 free base, WZ8040, Genistein (NPI 031L), Falnidamol, BLU-945, Sunvozertinib, CH7233163, Licochalcone D, Alflutinib (AST2818) mesylate, (Rac)-JBJ-04-125-02, Mobocertinib (TAK788), Tyrphostin AG30 (AG30), AG-1557, AG99, MTX-211, RG14620, Almonertinib (HS-10296), Cyasterone, Osimertinib mesylate, Norcantharidin, Naquotinib(ASP8273), EA1045, Lidocaine hydrochloride, Olmutinib (BI 1482694), Butein, Chrysophanic Acid, or (−)-Epigallocatechin Gallate, or a pharmaceutically acceptable salt thereof.

In some embodiments of a method of treating cancer, the additional agent is a VEGFR inhibitor.

In some embodiments of a method of treating cancer, the VEGFR inhibitor is Sorafenib (BAY 43-9006) tosylate, Sunitinib (SU11248) malate, Cabozantinib (BMS-907351), Ponatinib (AP24534), Axitinib (AG 013736), Foretinib (GSK1363089), Vandetanib (ZD6474), Nintedanib (BIBF 1120), Regorafenib (BAY 73-4506), Pazopanib HCl (GW786034 HCl), Cediranib (AZD2171), PD173074, Dovitinib (TKI-258), Linifanib (ABT-869), Vatalanib (PTK787) 2HCl, RAF265 (CHIR-265), Tivozanib (AV-951), Motesanib Diphosphate (AMG-706), Lenvatinib (E7080), Brivanib (BMS-540215), MGCD-265 analog, AEE788 (NVP-AEE788), ENMD-2076, OSI-930, CYC116, Ki8751, Telatinib, PP121, Pazopanib, KRN 633, SAR131675, Apatinib (YN968D1) mesylate, BMS-794833, Sorafenib (BAY 43-9006), Cabozantinib malate, Brivanib Alaninate (BMS-582664), Golvatinib (E7050), Semaxanib (SU5416), ZM 323881 HCl, ZM 306416, ENMD-2076 L-(+)-Tartaric acid, R1530, Chiauranib, Emvododstat (PTC299), XL092, Regorafenib Hydrochloride, Lucitanib (E3810) hydrochloride, Ningetinib, Donafenib (Sorafenib D3), Ki20227, Tyrphostin AG1433, SU14813, Sulfatinib, CS-2660 (JNJ-38158471), SU5204, SU5214, SU5205, SU5408, Pamufetinib (TAS-115), ODM-203, WHI-P180, Altiratinib, Motesanib (AMG-706), Fruquintinib (HMPL-013), Lenvatinib (E7080) Mesylate, Nintedanib Ethanesulfonate Salt, Apatinib, Cediranib Maleate, Toceranib phosphate, Anlotinib (AL3818) dihydrochloride, Regorafenib (BAY-734506) Monohydrate, Sitravatinib (MGCD516), Ramucirumab, BFH772, BAW2881 (NVP-BAW2881), SU5402, Sunitinib (SUl 1248), Dovitinib (TKI258) Lactate monohydrate, LY2874455, SKLB1002, AZD2932, Lenalidomide (CC-5013), WAY-340935, Oglufanide, hVEGF-IN-1, 4SC-203, Chebulinic acid, Nastorazepide, X-82 (Vorolanib), MAZ51, TMTD (Tetramethylthiuram disulfide), SU5208, SU5614, AG-13958, SKLB 610, SU1498, ZD-4190, PDGFR inhibitor 1, Bevacizumab, Erdafitinib (JNJ-42756493), Vitamin E, or Taxifolin (Dihydroquercetin), or a pharmaceutically acceptable salt thereof.

In some embodiments of a method of treating cancer, the additional agent is an aromatase inhibitor.

In some embodiments of a method of treating cancer, the aromatase inhibitor is Letrozole (CGS 20267), Anastrozole (ZD-1033), Exemestane (FCE 24304), Formestane, Fadrozole (CGS16949A), alpha-Naphthoflavone, or Obacunone (A13-37934), or a pharmaceutically acceptable salt thereof.

In some embodiments of a method of treating cancer, the additional agent is a mitotic inhibitor.

In some embodiments of a method of treating cancer, the mitotic inhibitor is a taxane (e.g., Paclitaxel and Docetaxel), a vinca alkaloid (e.g., Vinblastine, Vincristine, Vindesine, and Vinorelbine), Colchicine, Podophyllotoxin, Griseofulvin, or Glaziovianin A, or a pharmaceutically acceptable salt thereof.

In some embodiments of a method of treating cancer, the additional agent is a microtubule stabilizer.

In some embodiments of a method of treating cancer, the microtubule stabilizer is paclitaxel, nab-paclitaxel, docetaxel, colchicine, podophyllin, epothilone A, or epothilone B, or a pharmaceutically acceptable salt thereof.

In some embodiments of a method of treating cancer, the additional agent is a DNA cross-linker.

In some embodiments of a method of treating cancer, the DNA cross-linker is oxaliplatin, cisplatin, or a pharmaceutically acceptable salt thereof.

In some embodiments of a method of treating cancer, the additional agent is a vinca alkaloid.

In some embodiments of a method of treating cancer, the vinca alkaloid is vinorelbine, vincristine, vinblastine, vinblastine N-oxide, vindesine, vinflunine, vincamine, vintafolide, or deacetoxyvinzolidine, or a pharmaceutically acceptable salt thereof.

In some embodiments of a method of treating cancer, the additional agent is an alkylating agent.

In some embodiments of a method of treating cancer, the alkylating agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, platinum coordination complexes, or a pharmaceutically acceptable salt thereof.

In some embodiments of a method of treating cancer, the additional agent is folic acid analogs, pyrimidine analogs, purine analogs, antibiotics, L-asparaginase, interferons, anthracenedione substituted urea, methyl hydrazine derivatives, adrenocortical suppressant, adrenocorticosteroides, progestin, estrogen, antiestrogen receptor inhibitor, androgen, antiandrogen receptor inhibitor, endocrine/hormonal agents, or gonadotropin-releasing hormone analog, or a pharmaceutically acceptable salt thereof.

In some embodiments of a method of treating cancer, the antiestrogen receptor inhibitor is Fulvestrant (ICI-182780), Raloxifene HCl, Bazedoxifene (WAY-140424) HCl, G-1, Amcenestrant (SAR439859), Estrogen receptor modulator 1, Lasofoxifene Tartrate, H3B-5942, Raloxifene, Brilanestrant (GDC-0810), Tamoxifen (ICI 46474) Citrate, Toremifene Citrate (NK 622), Clomifene citrate, Estriol, Estrone, Bazedoxifene (TSE-424) acetate, SPP-86, Enclomiphene Citrate, Phenol Red sodium salt, Camizestrant (AZD9833), G15 (GRB-G15), Cyclofenil, PHTPP, AZD9496, Chlorotrianisene, Endoxifen HCl, Ospemifene, or Tamoxifen (ICI 46474), or a pharmaceutically acceptable salt thereof.

In some embodiments of a method of treating cancer, the antiandrogen receptor inhibitor is Enzalutamide (MDV3100), Bicalutamide (ICI-176334), Ostarine, Apalutamide (ARN-509), Galeterone, Flutamide, Cyproterone Acetate, AZD3514, Spironolactone, ORM-15341, CLP-3094, Proxalutamide (GT0918), JNJ-63576253 (TRC-253), Bavdegalutamide (ARV-110), ACP-105, Ailanthone, UT-34, Triptophenolide, 4,4′-DDE, EPI-001, Darolutamide (ODM-201), Megestrol Acetate, Clascoterone, Inobrodib (CCS-1477), GSK-2881078, (S,R,S)-AHPC (MDK7526), Dimethylcurcumin (ASC-J9), RU58841, Nilutamide, 3,3′-Diindolylmethane, or Chlormadinone acetate, or a pharmaceutically acceptable salt thereof.

In some embodiments of a method of treating cancer, the additional agent is a chemotherapeutic agent.

Also disclosed herein is a method of treating cancer in a subject in need thereof, the method comprising administering to the subject in need:
(a) 5-methoxy-1-(2-methylpyridin-3-yl)-4-(prop-2-yn-1-ylamino)-7-(trifluoromethyl)quinazolin-2(1H)-one:

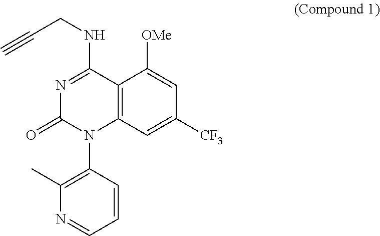 [SMILES: C#CCNc1nc(=O)n(-c2cccnc2C)c2cc(C(F)(F)F)cc(OC)c12]

or a pharmaceutically acceptable salt thereof; and
(b) docetaxel.

Also disclosed herein is a method of treating cancer in a subject in need thereof, the method comprising administering to the subject in need:
(a) 5-methoxy-1-(2-methylpyridin-3-yl)-4-(prop-2-yn-1-ylamino)-7-(trifluoromethyl)quinazolin-2(1H)-one:

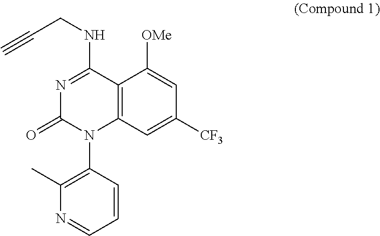 [SMILES: C#CCNc1nc(=O)n(-c2cccnc2C)c2cc(C(F)(F)F)cc(OC)c12]

or a pharmaceutically acceptable salt thereof; and
(b) radiation.

### Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, the compound of Formula (I) or Formula (II) (e.g., Compound 1) is administered in the range of 0.01 mg-5000 mg per day. In some embodiments, the compound of Formula (I) or Formula (II) (e.g., Compound 1) is administered from about 1 mg to about 1000 mg per day. In some embodiments, the desired dose is conveniently presented in a single dose or in divided doses administered simultaneously or at appropriate intervals, for example as two, three, four or more sub-doses per day. In some embodiments, the daily dosages appropriate for the compound described herein, or a pharmaceutically acceptable salt thereof, are from about 0.01 to about 50 mg/kg per body weight.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

### EXAMPLES

All compounds can be synthesized according to the methods disclosed in PCT/CN2022/126096, which is hereby incorporated by reference in its entirety.

The following examples are provided for illustrative purposes only and not to limit the scope of the claims provided herein.

### Intermediate C:

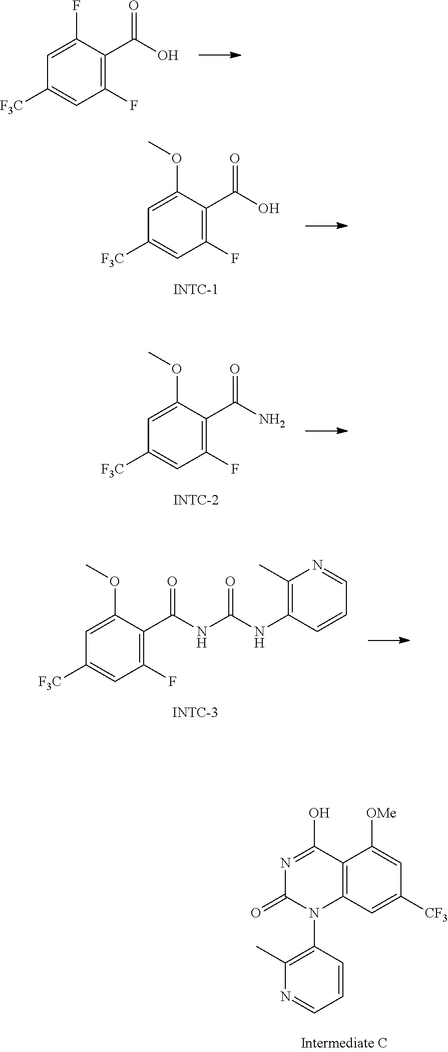

Step 1: To a solution of sodium methanolate (358.0 mg, 6.63 mmol) in MeOH (5 mL) were added 2,6-difluoro-4-(trifluoromethyl)benzoic acid (500.0 mg, 2.21 mmol), and the reaction was stirred at 80° C. for 2 hrs. The mixture was then cooled to room temperature and concentrated The residue was diluted with water (10 mL), adjusted pH to 2-3 with aqueous 6.0 M HCl and the mixture was extracted with DCM (15.0 mL×3), the organic layer was washed by brine, dried, and concentrated to afford compound INTC-1 (215.0 mg, 40.8% yield). ¹H NMR (400 MHz, DMSO-d₆) δ 7.37 (d, J=8.8 Hz, 1H), 7.28 (s, 1H), 3.92 (s, 3H).

Step 2: Compound INTC-1 (380.0 mg, 1.59 mmol) was mixed in SOCl₂ (6.0 mL), and then the resulting solution was heated at 80° C. for 2 hrs. The solution was then cooled to room temperature and concentrated to remove excess SOCl₂. The residue was taken up in dioxane (6.0 mL), treated with NH₄OH (40% w/w, 6.0 mL). The resulting solution was stirred at 0° C. to room temperature for 1 h. The mixture was concentrated under reduced pressure and diluted with water. Then the solid product formed was collected and washed with water and dried to afford compound INTC-2 (230 mg, 60.8% yield). ¹H NMR (400 MHz, DMSO-d₆) δ 7.96 (s, 1H), 7.71 (s, 1H), 7.31 (dd, J=8.8 Hz, 0.4 Hz, 1H), 7.23 (s, 1H), 3.89 (s, 3H).

Step 3: To a stirred suspension of compound INTC-2 (230 mg, 0.97 mmol) in dichloroethane (5 mL) at room temperature was added oxalyl dichloride (135 mg, 1.07 mmol). The resultant suspension was heated to 80° C. for 1 h. The mixture was cooled to room temperature, 2-methylpyridin-3-amine (209.8 mg, 1.94 mmol) was added. The mixture was stirred at room temperature for 16 hrs. The precipitate was collected, washed with water, and dried to afford compound INTC-3 (120 mg, 33.3% yield). LCMS: 372.0 [M+H]⁺.

Step 4: KHMDS (0.47 mL, 0.47 mmol, 1.0 M in THF) was added to a mixture of compound INTC-3 (80.0 mg, 0.21 mmol) in THF (3.0 mL) at −20° C., and the resulting mixture was allowed to warm to room temperature over 1 h. The mixture was concentrated, diluted with water, and adjusted pH to 6-7 with aqueous 4.0 M HCl. The precipitate was collected, washed with water, and dried to afford compound Intermediate C (60.0 mg, 79.2% yield). ¹H NMR (400 MHz, DMSO-d₆) δ 11.71 (s, 1H), 8.66 (dd, J=4.8 Hz, 1.6 Hz, 1H), 7.88 (dd, J=8.0 Hz, 1.6 Hz, 1H), 7.52 (dd, J=7.6 Hz, 4.8 Hz, 1H), 7.15 (s, 1H), 5.98 (s, 1H), 3.98 (s, 3H), 2.25 (s, 3H).

### Example 36: Synthesis of 5-methoxy-1-(2-methylpyridin-3-yl)-4-(prop-2-yn-1-ylamino)-7-(trifluoromethyl)quinazolin-2(1T)-one (Compound 1)

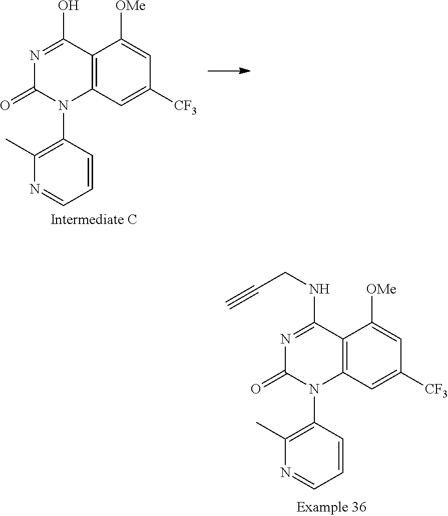 [SMILES: COc1cc(C(F)(F)F)cc2c1c(O)nc(=O)n2-c1cccnc1C ; C#CCNc1nc(=O)n(-c2cccnc2C)c2cc(C(F)(F)F)cc(OC)c12]

To a stirred suspension of compound Intermediate C (70.0 mg, 0.20 mmol) in toluene (1 mL) was added DIPEA (257.0 mg, 1.99 mmol) and POCl₃ (153.0 mg, 1.00 mmol) at 0° C. The resultant suspension was heated at 100° C. for 2 hrs. After cooling to room temperature, a solution of DIPEA (257.0 g, 1.99 mmol) and prop-2-yn-1-amine (109.6 mg, 1.99 mmol) in NMP (0.5 mL) was added. The mixture was stirred at 50° C. for 1 h. Water (10 mL) was added, extracted by dichloromethane (5 mL×3), the organic layer was washed by brine, dried over anhydrous Na₂SO₄, filtered, concentrated, and purified by column chromatography to give example 36 (29.0 mg, 37.9% yield). LCMS: 389.1 [M+H]⁺. ¹H NMR (400 MHz, DMSO-d₆) δ 8.96 (t, J=5.2 Hz, 1H), 8.61 (dd, J=4.8, 1.6 Hz, 1H), 7.75 (dd, J=8.0, 1.6 Hz, 1H), 7.47 (dd, J=7.6, 4.8 Hz, 1H), 7.15 (s, 1H), 6.03 (s, 1H), 4.39-4.26 (m, 2H), 4.13 (s, 3H), 3.16 (t, J=2.4 Hz, 1H), 2.16 (s, 3H)

### Example A: MAT2A Biochemical Assay

Compounds described herein were tested as follows:

### Enzyme Reaction

(1). Prepared 1×Assay buffer.
(2). Preparation of compound concentration gradient: the compounds test condition were 1 p M start, 3-fold dilution, 10 doses, singlet or duplicate. 100x concentration compounds were prepared in 384-well plate. Then used Echo 550 to transfer 250 nl to a 384-reaction plate for later use. Added 250 nl of 100% DMSO to the negative and positive control wells.
(3). Prepared 1.67× final concentration enzyme solution with 1×Assay buffer.
(4). Added 15 μl of 1.67× Enzyme solution to the compound wells and positive control wells; added 15 μl of 1×Assay buffer to the negative control wells.
(5). Centrifuged at 1000 rpm for 30 seconds and incubate for 15 minutes.
(6). Prepared 2.5× final concentration Substrate mix solution with 1×Assay buffer.
(7). Added 10 μl of 2.5× final concentration Substrate mix solution to start the reaction.
(8). Centrifuged at 1000 rpm for 30 seconds and incubate for 150 minutes.
(9). Added 50 pl Biomol Green to stop the reaction, centrifuge at 1000 rpm for 30 seconds and incubate for 15 minutes. read O.D.620, process data.

### Data Analysis

(1) Using GraphPad Prism 5.

$$\% Inh = ( Max signal - Compound signal ) / ( Max signal - Min signal ) * 100. \qquad ( 2 )$$

(3) Max signal was obtained from the positive control wells.
(4) Min signal was obtained from the negative control wells.

The data from Example A is shown in Table 6.

TABLE 6

| Example # | MAT2A enzymatic IC₅₀ (nM) |
|---|---|
| 1 | A |
| 2 | A |
| 3 | B |
| 4 | A |
| 5 | A |
| 6 | A |
| 7 | A |
| 8 | A |
| 9 | C |
| 10 | A |
| 11 | C |
| 12 | C |
| 13 | E |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | B |
| 33 | A |
| 34 | A |
| 35 | A |
| 36 (Compound 1) | A |
| 37 | A |
| 38 | A |
| 39 | B |
| 40 | A |
| 41 | B |
| 42 | A |
| 43 | B |
| 44 | C |
| 45 | A |
| 46 | A |
| 47 | A |
| 48 | A |
| 49 | A |
| 50 | A |
| 51 | A |
| 52 | A |
| 53 | A |
| 54 | A |
| 55 | D |
| 56 | E |
| 57 | A |
| 58 | C |
| 59 | C |
| 60 | C |
| 61 | A |
| 62 | B |
| 63 | A |
| 64 | A |
| 65 | A |
| 66 | A |
| 67 | A |
| 68 | A |
| 69 | A |
| 70 | A |
| 71 | C |
| 72 | C |
| 73 | A |
| 74 | A |
| 75 | A |
| 76 | A |
| 77 | A |
| 78 | B |
| 79 | C |

IC₅₀ (nM): 0 < A ≤ 50; 50 < B ≤ 100; 100 < C ≤ 500; 500 < D ≤ 1,000; 1,000 < E

### Example B: In Vivo Efficacy of an MAT2A Inhibitor Combined with a Chemotherapy

### Efficacy Study of KP-4 Human Pancreatic Cancer Xenograft Model in NOD SCID Mice

### Methods:

### Cell Culture

The KP-4 tumor cell line was maintained in vitro as monolayer culture in RPMI 1640 medium supplemented with 10% fetal bovine serum at 37° C. in an atmosphere of 500 CO₂ in air. The tumor cells were routinely subculture weekly by trypsin-EDTA treatment, not to exceed 4-5 passages. The cells growing in an exponential growth phase was harvested and counted for tumor inoculation.

### Method for Tumor Inoculation and Randomization

Each mouse was inoculated subcutaneously on the central right flank with KP-4 tumor cells (1×10⁷) in 0.1 mL of RPMI-1640 without serum for the tumor development. The treatment started when the mean tumor size reached approximately 100 mm³. Mice were assigned to groups such that the mean tumor volume is the same for each treatment group and time point.

### Measurement Parameters

For routine monitoring, all study animals were monitored for not only tumor growth but also behavior such as mobility, food, and water consumption (by cage side checking only), body weight (BW), eye/hair matting and any other abnormal effect. Any mortality and/or abnormal clinical signs were recorded.

### Body Weight

Body weights of all animals were measured twice a week throughout the study. Body weight change, expressed in %, will be calculated using the following formula: BW change (%)=(BW_{Day x}/BW_{Day 0})×100, where BW_{Day x} is BW on a given day, and BW_{Day 0} is BW on Day 0 (initiation of treatment).

### Tumor Measurements

The measurement of tumor size were conducted twice a week with a caliper and the tumor volume (mm³) was estimated using the formula: TV=a×b²/2, where a and b are long and short diameters of a tumor, respectively. The TVs were used for calculation of the tumor growth inhibition (TGI, an indicator of antitumor effectiveness) value using the formula: TGI (%)=[1−(Tₙ−T₀)/(Cₙ−C₀)]×100%, only over the dosing period (dosing days 0 to days n). Where: Tₙ—is the avg tumor volume at the respective day “n” after dosing throughout treatment period; T₀—is the avg tumor volume in the treatment group at day 0 before treatment (immediately before); Cₙ—avg tumor volume in the control group at the respective day “n” after dosing throughout treatment period; and C₀—average tumor volume in the control group at day 0 before treatment (immediately before). The experiment was terminated when the mean tumor volume exceeded 2000 mm³ or severe body weight loss.

### Statistical Analysis

The statistical software GraphPad Prism (version number: 8) was used to analyze the difference in tumor volume between the treatment group and the solvent control group. Two-way ANOVA and Bonferroni multiple tests were used to compare whether there was a significant difference in tumor volume between the vehicle control group and each treatment group during the administration period. Using One-way ANOVA and Dunnett's multiple comparisons to analyze whether there is a significant difference in the tumor volume between the vehicle control group and each treatment group at 21 days after administration, and whether there is a difference in the tumor weight between the vehicle control group and each treatment group at the end of the experiment, *P<0.05, **P<0.01, ***P<0.001 considered the data to be significantly statistically different.

### Results

Mean tumor volume at dosing start was approximately 102 mm³, with ten mice randomized to each treatment group. In the KP4 MTAP null tumor model, mice were dosed orally, once per day (QD) with Vehicle, once per day (QD) with Compound 1 at 1, 3 mg/kg, once per day (QD) with AG270 at 100 mg/kg, mice were intravenous, per week with Docetaxel at 2.5 mg/kg, or Compound 1 combined with Docetaxel at each dose level.

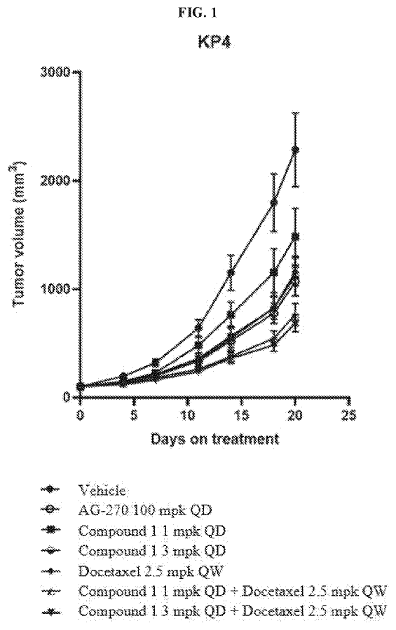

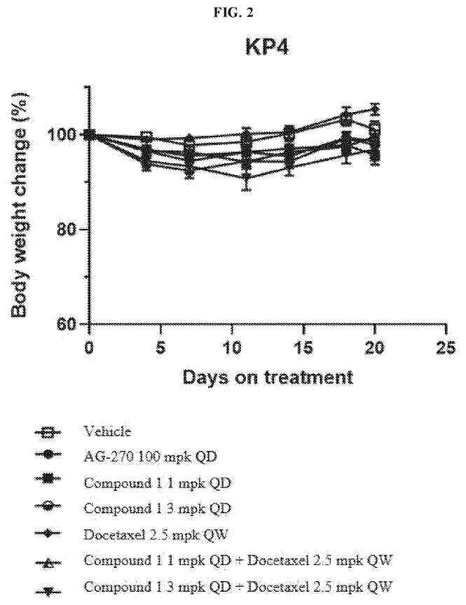

In the KP-4 human pancreatic cancer xenograft model, administration of Compound 1 at 1 or 3 mg/kg QD resulted in 36.8% o and 53.4% o TGI, respectively. Administration of AG270 at 100 mg/kg resulted in 55.7 TGI. Administration of Docetaxel at 2.5 mg/kg resulted in 51.6% TGI. The combination of 2.5 mg/kg Docetaxel per week and Compound 1 at 1, 3 mg/kg resulted in 69.600 and 73.200 TGI, respectively (Table 7 and FIG. 1). The combination of Compound 1 and Docetaxel significantly inhibited tumor growth in a dose dependent manner. All mice were well tolerated, mice weight change showed in FIG. 2.

TABLE 7
summary of efficacy of Compound 1, AG270 and
Docetaxel in KP4 MTAP null xenografts.

| | | | | Tumor volume (mm³)^{a} | | | |
|---|---|---|---|---|---|---|---|
| | | | Dose | Initial | Final | TGI | |
| Group | Treatment | number | (mg/kg) | (mean) | (mean) | (%) | P value ^{b} |
| 1 | Vehicle | 10 | — | 102 ± 3 | 2,287 ± 340 | — | — |
| 2 | AG270 | 10 | 100 | 102 ± 4 | 1,070 ± 132 | 55.7 | 0.003 |
| 3 | Compound 1 | 10 | 1 | 102 ± 3 | 1,484 ± 263 | 36.8 | 0.096 |
| 4 | Compound 1 | 10 | 3 | 102 ± 3 | 1,121 ± 181 | 53.4 | 0.007 |
| 5 | Docetaxel | 10 | 2.5 | 102 ± 3 | 1,159 ± 129 | 51.6 | 0.004 |
| 6 | Compound 1 | 10 | 1 | 102 ± 3 | 767 ± 101 | 69.6 | <0.001 |
| | Docetaxel | | 2.5 | | | | |
| 7 | Compound 1 | 10 | 3 | 102 ± 3 | 686 ± 80 | 73.2 | <0.001 |
| | Docetaxel | | 2.5 | | | | |

^{a}Mean ± SD;
^{b} Comparison with Vehicle, One-way ANOVA

### Example C: In Vivo Efficacy of an MAT2A Inhibitor Combined with a Chemotherapy

### Efficacy Study of HCC15 Human Lung Cancer Xenograft Model in NOD SCID Mice Methods:

### Cell Culture:

The HCC15 cells were maintained in vitro as a monolayer culture in 90% 1640+10% FBS, 100U/ml penicillin and 100 μg/ml streptomycin at 37° C. in an atmosphere of 5% CO2 in air. The tumor cells were routinely subcultured at a ratio of 1:2 to 1:3 every 3-4 days. Alternatively, Cultures were maintained by addition or replacement of fresh medium. Started cultures at 5×10\hat{ }5 cells/mL and maintained between 0.5×10\hat{ }5 and 2×10\hat{ }6 cells/ml.

### Animals:

NOD SCID mice, female, 6-8 weeks, weighing approximately 20-22 g.

### Tumor Inoculation:

Each mouse were inoculated subcutaneously at the right flank with HCC15 cells in a 0.2 mL mixture of 1640 for tumor development (5×10⁶ cells/mouse). Dosing was started when the average tumor size reaches approximately 100-200 mm³ for the tumor efficacy study with tool and med chem molecules to look for efficacy vs vehicle tumor growth.

### Results

Mean tumor volume at dosing start was approximately 133.09 mm³, with ten mice randomized to each treatment group. In the HCC15 MTAP null tumor model, mice were dosed orally, once per day (QD) with Vehicle, once per day (QD) with Compound 1 at 3 mg/kg, once per day (QD) with AG270 at 100 mg/kg, mice were intravenous, per week with Docetaxel at 2.5 mg/kg, or Compound 1 combined with Docetaxel at each dose level.

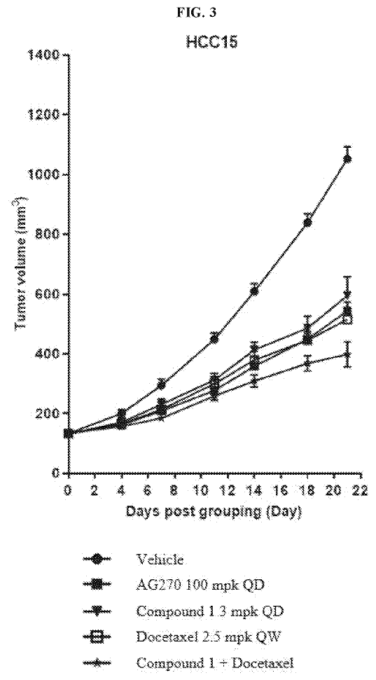

In the HCC15 Human Lung Cancer Xenograft Model, administration of Compound 1 at 3 mg/kg QD resulted in 44.6% TGI, respectively. Administration of AG270 at 100 mg/kg resulted in 51.5% TGI. Administration of Docetaxel at 2.5 mg/kg resulted in 51.8% TGI. The combination of 2.5 mg/kg Docetaxel per week and Compound 1 at 3 mg/kg resulted in 64.2% TGI, respectively (Table 8 and FIG. 3). The combination of Compound 1 and Docetaxel significantly inhibited tumor growth.

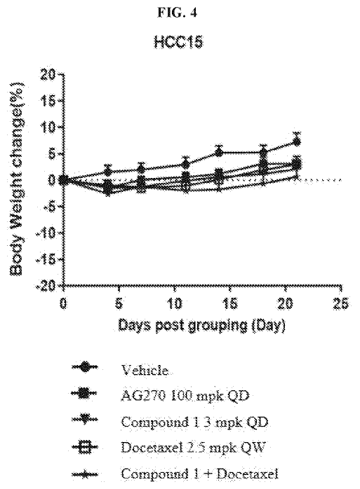

All mice were well tolerated, mice weight change showed in FIG. 4.

TABLE 8
summary of efficacy of Compound 1, AG270 and
Docetaxel in HCC15 MTAP null xenografts.

| | | | | Tumor volume (mm³)^{a} | | | |
|---|---|---|---|---|---|---|---|
| | | | Dose | Initial | Final | TGI | |
| Group | Treatment | number | (mg/kg) | (mean) | (mean) | (%) | P value ^{b} |
| 1 | Vehicle | 10 | — | 134 ± 7 | 1053 ± 41 | — | — |
| 2 | AG270 | 10 | 100 | 133 ± 6 | 544 ± 30 | 55.3 | <0.001 |
| 3 | Compound 1 | 10 | 3 | 133 ± 5 | 596 ± 63 | 49.6 | <0.001 |
| 4 | Docetaxel | 10 | 2.5 | 133 ± 6 | 515 ± 36 | 58.5 | <0.001 |
| 5 | Compound 1 | 10 | 3 | 135 ± 6 | 399 ± 42 | 71.2 | <0.001 |
| | Docetaxel | | 2.5 | | | | |

^{a}Mean ± SD;
^{b} Comparison with Vehicle, One-way ANOVA

### Example D: In Vivo Efficacy of an MAT2A Inhibitor Combined with a PRMT5 Inhibitor

### Efficacy Study of NCI-H838 Human Lung Cancer Xenograft Model in NOD SCID Mice Methods:

### Cell Culture:

The NCI-H838 cancer cells were maintained in vitro with RPMI-1640 medium supplemented with 10% fetal bovine serum at 37TC in an atmosphere of 5% CO2 in the air. The cells in exponential growth phase were harvested and quantitated by cell counter before tumor inoculation.

### Animals:

NOD SCID mice, female, 6-8 weeks, weighing approximately 20-22 g, All the mice were purchased from Shanghai Lingchang Bio-Tech Co., Ltd.

### Tumor Inoculation:

Each mouse was inoculated subcutaneously at the right flank with NCI-H838 cells in a 0.1 mL of PBS mixed with Matrigel (1:1) for tumor development (5×10⁶ cells/mouse). Dosing was started when the average tumor size reached approximately ~150 mm³ for the tumor efficacy study with tool and med chem molecules to look for efficacy vs vehicle tumor growth.

### Results

Mean tumor volume at dosing start was approximately 146.51 mm³, with six mice randomized to each treatment group. In the NCI-H838 MTAP null tumor model, mice were dosed orally, once per day (QD) with vehicle, once per day (QD) with Compound 1 at 3, 10 mg/kg, mice were intravenous, once per day (QD) with PRMT5 inhibitor MRTX1719 at 50 mg/kg, or Compound 1 combined with MRTX1719 at each dose level.

In the NCI-H838 human lung cancer xenograft model, administration of Compound 1 at 3 or 10 mg/kg QD resulted in 1.6% and 19.9% TGI, respectively. Administration of MRTX1719 at 50 mg/kg resulted in 81.5% TGI. The combination of 50 mg/kg MRTX1719 and Compound 1 at 3, 10 mg/kg resulted in 83.2% and 103.1% TGI, respectively (Table 9 and FIG. 5). The combination of Compound 1 and MRTX1719 significantly inhibited tumor growth and showed significantly additive or synergistic effect which synergy effect is 1.02 and 1.21, respectively. The administrations were well tolerated. Mice weight changes are shown in FIG. 6.

TABLE 9
summary of efficacy of Compound 1 and MRTX1719
in NCI-H838 MTAP null xenografts.

| | | | | Tumor volume (mm³)^{a} | | | |
|---|---|---|---|---|---|---|---|
| | | | Dose | Initial | Final | TGI | |
| Group | Treatment | number | (mg/kg) | (mean) | (mean) | (%) | P value ^{b} |
| 1 | Vehicle | 6 | — | 147 ± 6 | 2059 ± 300 | — | — |
| 2 | Compound 1 | 6 | 3 | 146 ± 6 | 2029 ± 239 | 1.6 | <0.001 |
| 3 | Compound 1 | 6 | 10 | 146 ± 6 | 1678 ± 113 | 19.9 | <0.001 |
| 4 | MRTX1719 | 6 | 50 | 147 ± 6 | 501 ± 69 | 81.5 | <0.001 |
| 5 | Compound 1 | 6 | 3 | 147 ± 5 | 468 ± 115 | 83.2 | <0.001 |
| | MRTX1719 | | 50 | | | | |
| 6 | Compound 1 | 6 | 10 | 147 ± 6 | 88 ± 7 | 103.1 | <0.001 |
| | MRTX1719 | | 50 | | | | |

Note:
^{a}Mean ± SD;
^{b} Comparison with Vehicle, One-way ANOVA

### Example E: Synergy Screen of MAT2A Inhibitor and Cancer Therapeutic Agents

A synergistic screen was performed to evaluate the potential synergistic effects of MAT2A inhibitor (Compound 1) in combination with other therapeutic agents for cancer treatment. These agents included PARP inhibitors (e.g. olaparib, talazoparib), mTOR inhibitors (e.g. everolimus, temsirolimus), antimetabolites (e.g. decitabine, pemetrexed), CDK4/6 inhibitors (e.g. abemaciclib, palbociclib), BCL-2 inhibitor (e.g. venetoclax), an alkylating agent (e.g. oxaliplatin, altretamine), microtubule-stabilizing agents (e.g. docetaxel), vinca alkaloid (e.g. vinorelbine), KRAS inhibitor (e.g. sotorasib), EGFR inhibitors (e.g. afatinib, gefitinib), topoisomerase inhibitors (e.g. etoposide), PRMT5 inhibitors (e.g. MRTX1719, AM-9747), WEE1 inhibitor (e.g. bosutinib), and hypomethylating agent (e.g. procainamide). In a dose matrix, a series of dilutions of MAT2A inhibitor (Compound 1) and combination agents were prepared. And a panel of 43 cancer cell lines (Table 10) was treated to evaluate the potential synergistic effects on cell growth inhibition.

### Drug Combination Treatment and Cell Viability Assays

Cells were placed at the optimal seeding density per well in 384-well plates. Serially diluted (1:3 dilution, 10 μM as the highest concentration) Compound 1 and other cancer therapeutic agents in a 6×6 matrix were added after seeding. Assay plates were then incubated for 7 or 10 days followed by addition of 25 μl of CellTiter-Glo (Promega Corp.) at room temperature. Plates were read on the Envision (PerkinElmer Inc.) using enhanced luminescence protocol.

### Excess Over Bliss as a Measurement for Synergy

Combination synergy was assessed using the Bliss independence model. At designated doses of the component drugs, if compound A and B, with experimentally measured fractional inhibitions EA and EB, then the expected fractional inhibition, EAB, induced by their combination should be calculated using EA+EB−EA×EB. Excess over Bliss score (EOB) was determined by computing the difference in fractional inhibition induced by compound combination, EZ, and the expected fractional inhibition, EAB, i.e., EOB=EZ−EAB. In our study, Excess over Bliss scores were only calculated for combinations eliciting >20% growth inhibition. Scores between 0 and 10 were considered additive, scores >10 were considered synergistic.

TABLE 10
Cancer cell lines used to evaluate potential synergy between
Compound 1 and other cancer therapeutic agents

| Tissue | Names | MTAP Status |
|---|---|---|
| Bladder | RT4 | Null |
| Bladder | RT112-84 | Null |
| Breast | HCC38 | Null |
| Central Nervous System | SW1088 | Null |
| Liver | SK-HEP-1 | Null |
| Liver | huH-1 | Wild type |
| Lung | SW900 | Null |
| Lung | SW1573 | Null |
| Lung | NCI-H838 | Null |
| Bladder | SW780 | Null |
| Bladder | UM-UC-3 | Null |
| Breast | MCF-7 | Null |
| Breast | HCC1395 | Null |
| Breast | MDA-MB-231 | Null |
| Breast | HCC1806 | Null |
| Breast | BT-474 | Wild type |
| Breast | MFM-223 | Wild type |
| Central Nervous System | U-87-MG | Null |
| Central Nervous System | U-138-MG | Null |
| Central Nervous System | A172 | Null |
| Haematopoietic and | MV-4-11 | Wild type |
| Lymphoid | | |
| Haematopoietic and | DOHH-2 | Null |
| Lymphoid | | |
| Haematopoietic and | K-562 | Null |
| Lymphoid | | |
| Haematopoietic and | RS4-11 | Null |
| Lymphoid | | |
| Haematopoietic and | Jurkat | Null |
| Lymphoid | | |
| Haematopoietic and | THP-1 | Null |
| Lymphoid | | |
| Haematopoietic and | MOLM-13 | Wild type |
| Lymphoid | | |
| Haematopoietic and | Toledo | Wild type |
| Lymphoid | | |
| Large Intestine | HCT-116 MTAP-null | Knock out |
| Large Intestine | NCI-H716 | Wild type |
| Lung | NCI-H292 | Null |
| Lung | A549 | Null |
| Lung | NCI-H1651 | Null |
| Lung | NCI-H1792 | Wild type |
| Pancreas | KP-4 | Null |
| Pancreas | LN-18 | Null |
| Pancreas | MIA-PaCa-2 | Null |
| Pancreas | CAPAN-1 | Null |
| Pancreas | PANC-1 | Null |
| Pancreas | AsPC-1 | Wild type |
| Pancreas | HuP-T4 | Null |

### Results

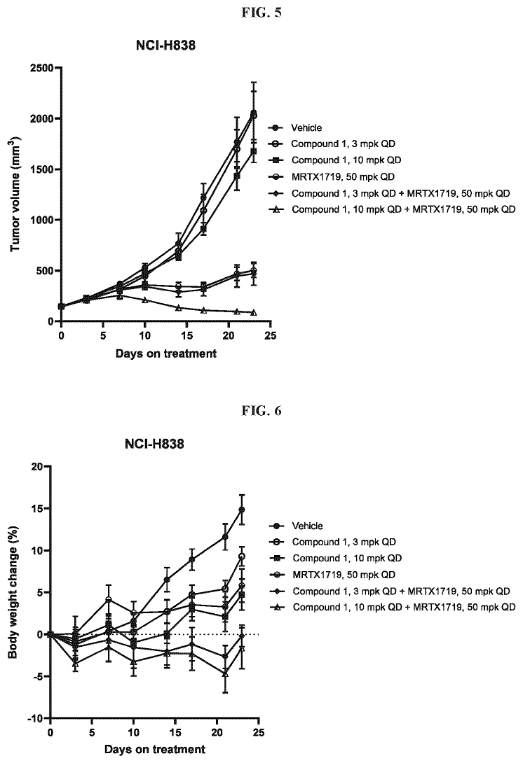

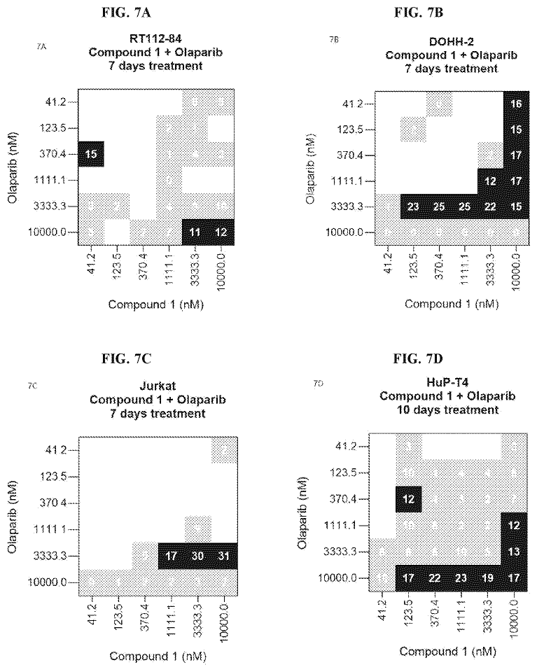

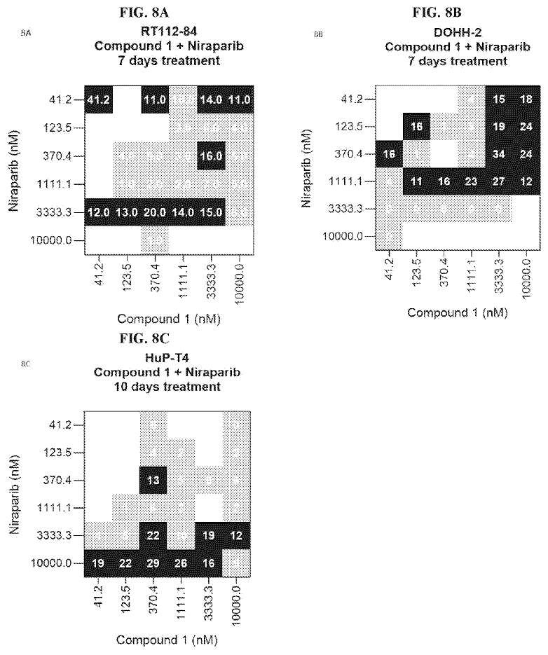

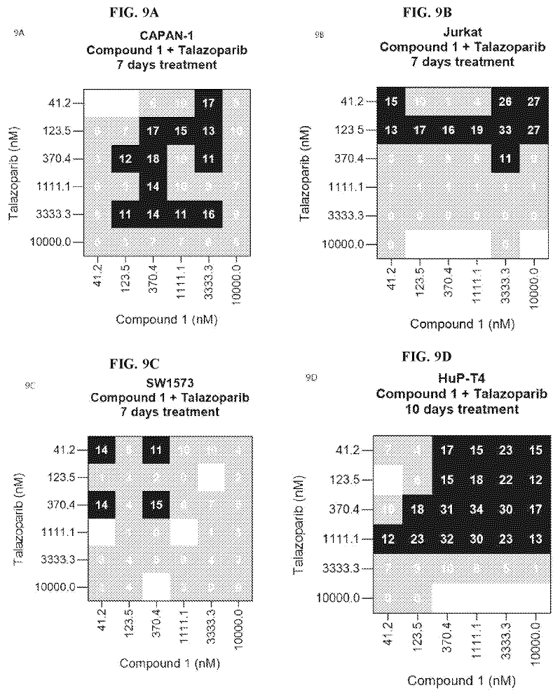

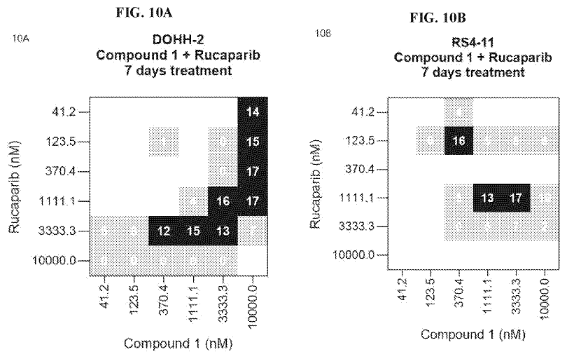

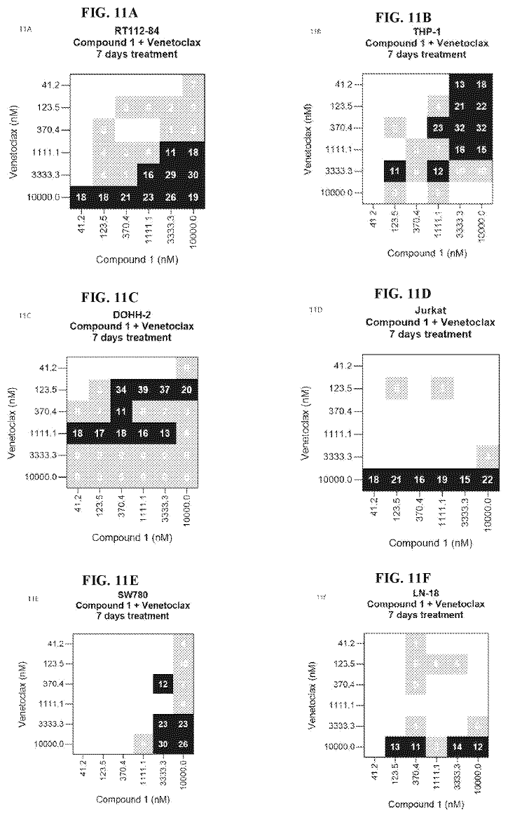

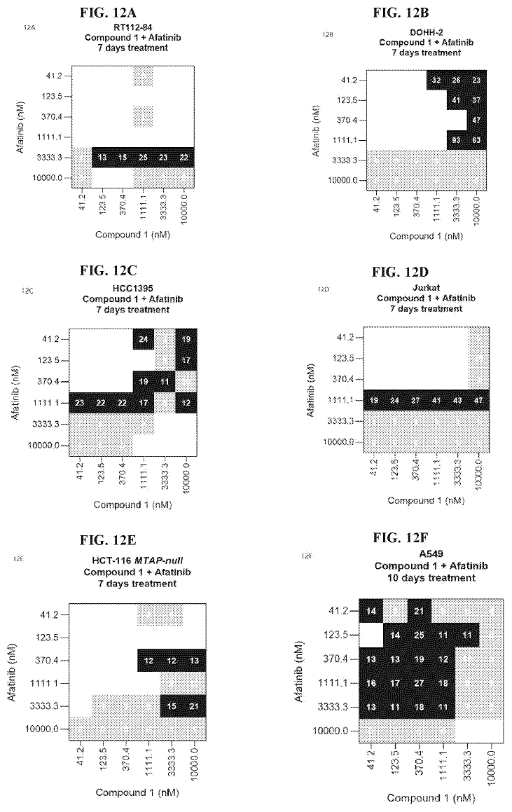

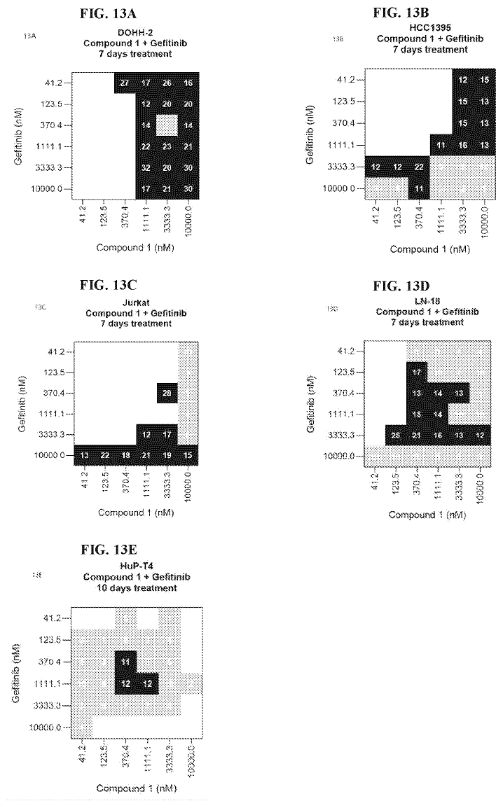

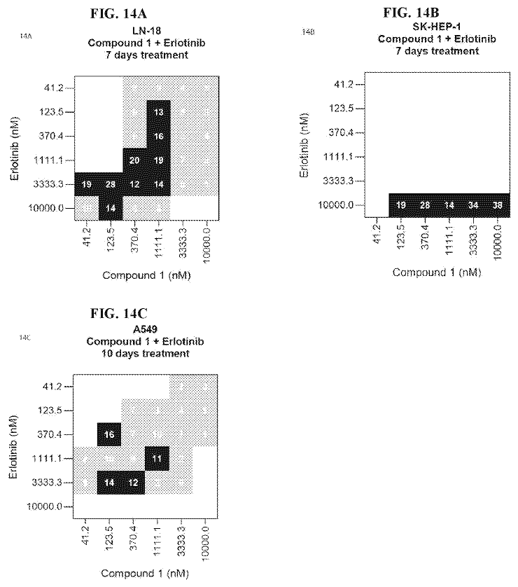

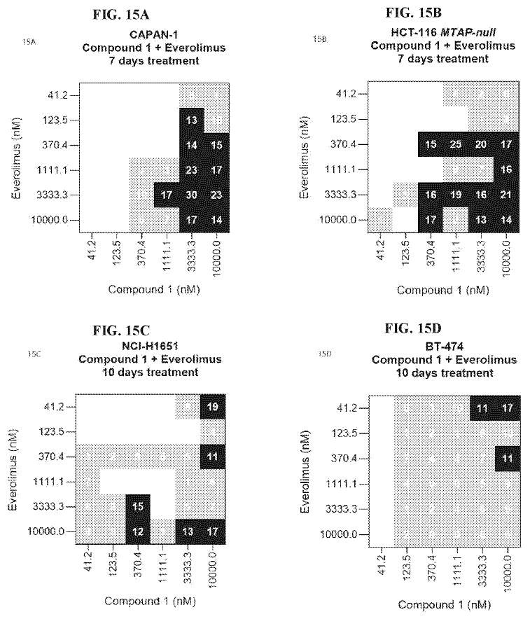

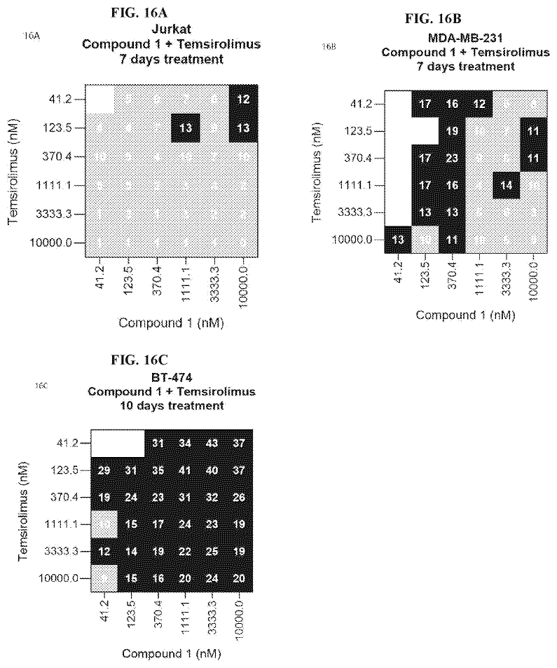

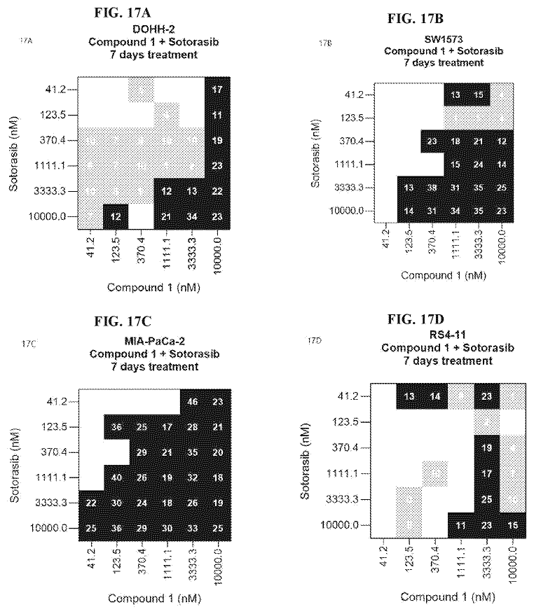

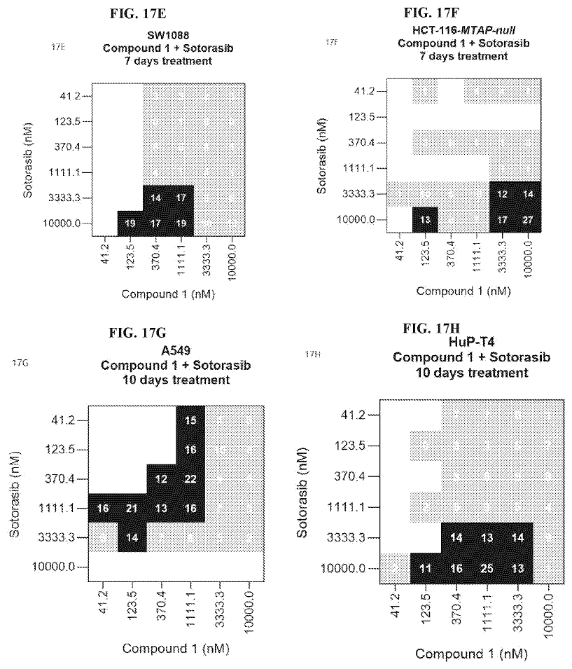

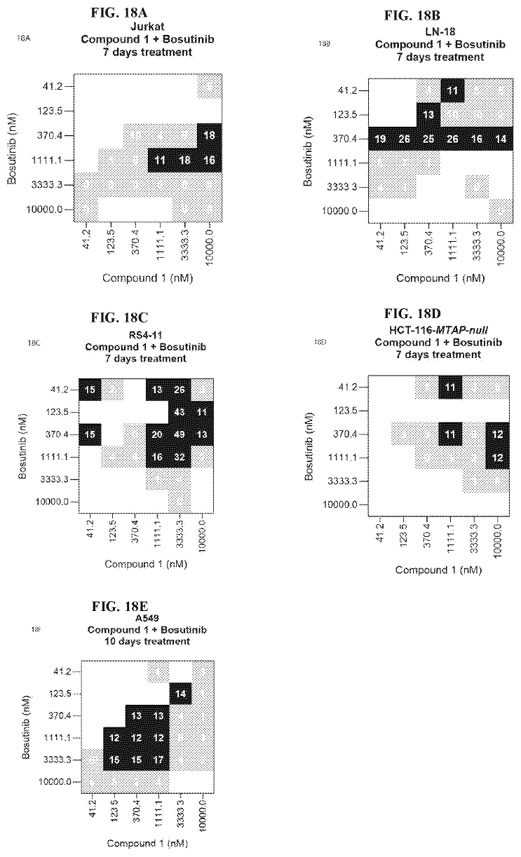

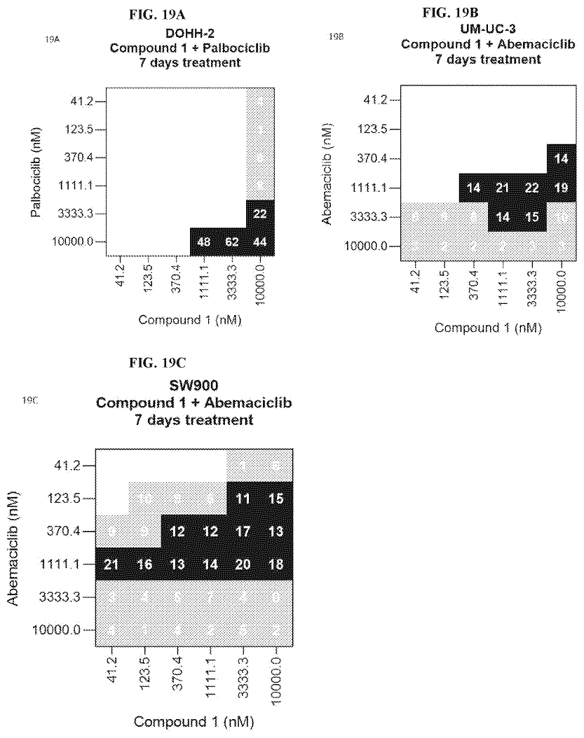

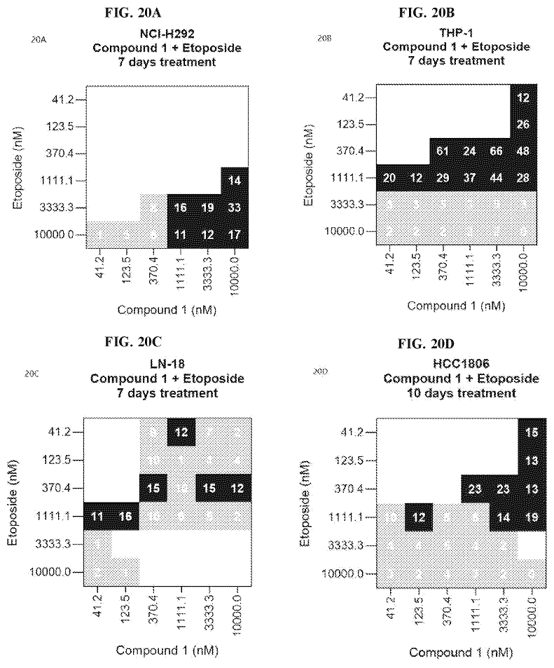

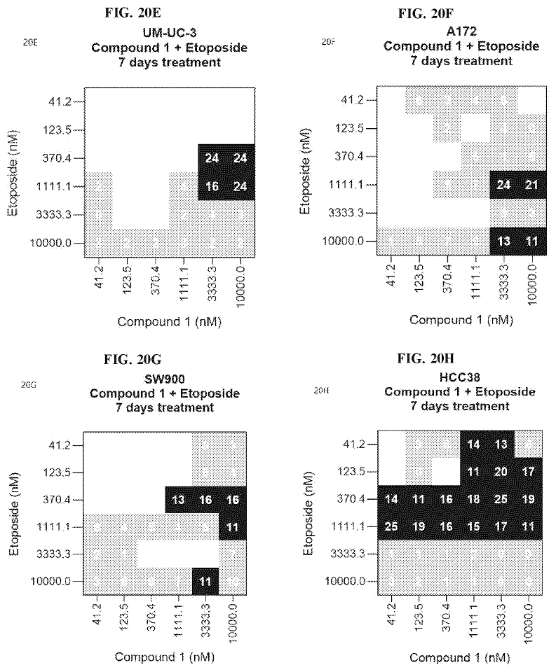

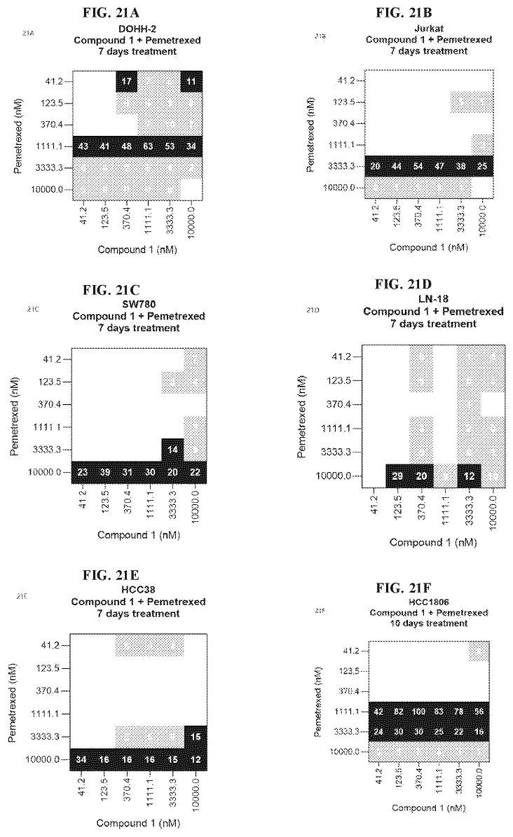

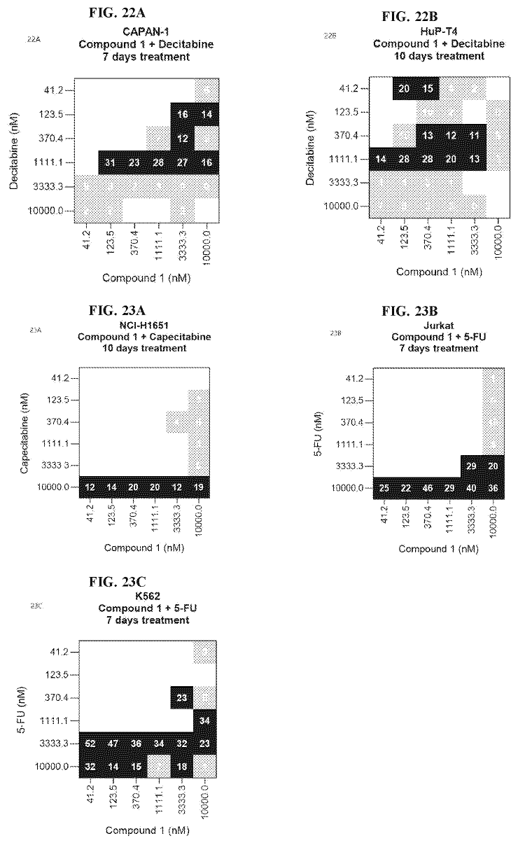

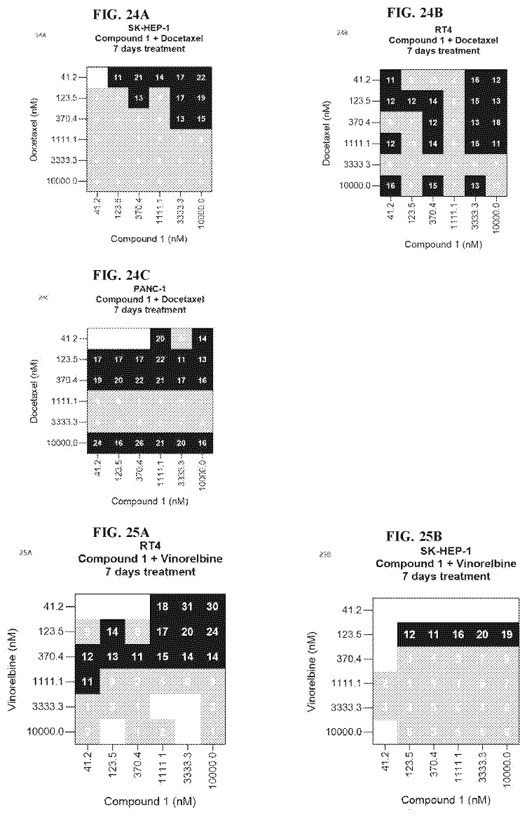

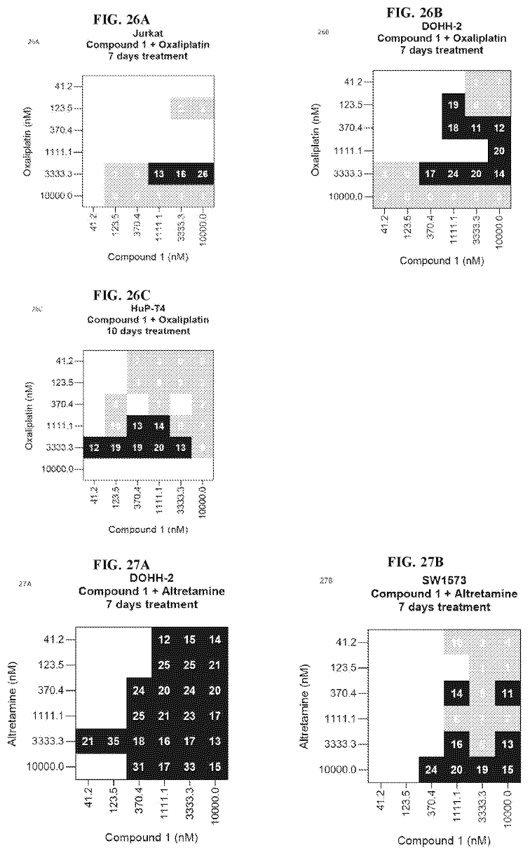

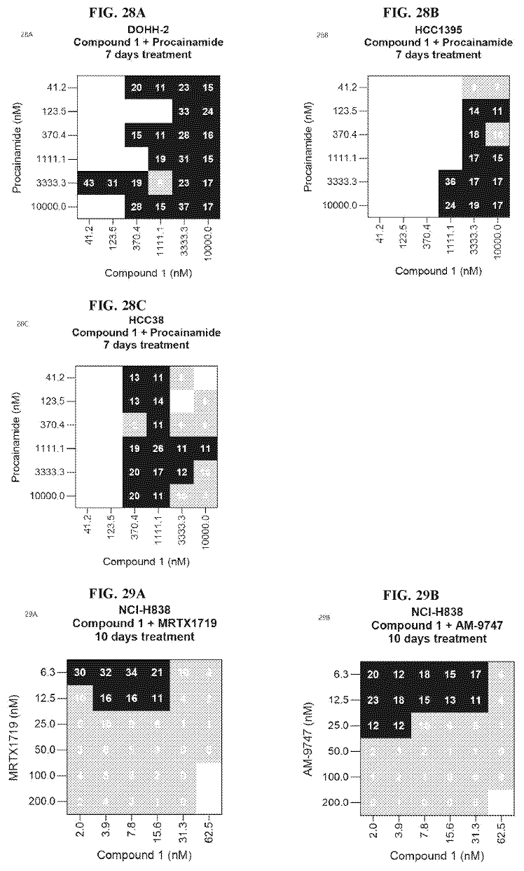

Synergistic growth inhibition between Compound 1 and several cancer therapeutic agents (PARP inhibitor, BCL2 inhibitor, EGFR inhibitor, mTOR inhibitor, KRAS inhibitor, PRMT5 inhibitor, protein kinase inhibitor, dual SRC and ABL tyrosine kinase inhibitor, CDK4/6 inhibitor, topoisomerase inhibitor, antimetabolites, microtubule-stabilizing agent, microtubule-destabilizing agent, and DNA cross-linking agent) was observed in multiple MTAP-deficient and MTAP-knock-out cell lines (FIG. 7A-7D, 8A-8C, 9A-9D, 10A-10B, 11A-11F, 12A-12F, 13A-13E, 14A-14C, 15A-15D, 16A-16C, 17A-17H, 18A-18E, 19A-19C, 20A-20H, 21A-21F, 22A-22B, 23A-23C, 24A-24C, 25A-25B, 26A-26C, 27A-27B, 28A-28C, and 29A-29B). Synergy was determined across all dosing levels using Excess over Bliss. Additive growth inhibition (scores between 0-10) was highlighted light gray and synergy growth inhibition (scores >10) was highlighted black. Beyond combination synergy, additive growth inhibition was observed in MTAP-deficient and MTAP-knock-out cell lines.

What is claimed is:

1. A method of treating cancer in a subject in need thereof, the method comprising administering to the subject:
(a) a compound of Formula (I) or Formula (II), or a pharmaceutically acceptable salt thereof:
wherein: 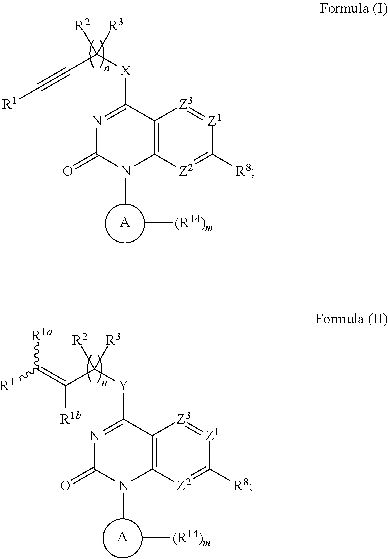
Â is selected from C₃₋₆cycloalkyl, C₂₋₉heterocycloalkyl, C₆₋₁₀aryl, and C₁₋₁₀heteroaryl;
Z¹ is CR⁷ or N;
Z² is CR⁹ or N;
Z³ is CR⁶ or N;
Z⁴ is CR^{6a} or N;
X is selected from —N(R⁴)—, —O—, and —C(R⁵)(R^{5a})—;
Y is selected from —N(R^{4a})—, —O—, and —C(R⁵)(R^{5a})—;
R¹ is selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15a};
R^{1a} and R^{1b} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15a};
each R² and each R³ are each independently selected from hydrogen, halogen, oxo, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉ heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15b}; or R² and R³, together with the carbon to which they are attached, form a C₃₋₆cycloalkyl or C₂₋₉heterocycloalkyl;
R⁴ is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R⁴ and an R³ are combined to form a C₂₋₉heterocycloalkyl optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
R^{4a} is selected from hydrogen, C₁₋₆alkyl, C₁₋₆haloalkyl, C₂₋₆alkenyl, C₂₋₆alkynyl, C₃₋₆cycloalkyl, C₂₋₉ heterocycloalkyl, C₆₋₁₀aryl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R^{4a} and an R³ are combined to form a C₂₋₉heterocycloalkyl optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
R⁵ and R^{5a} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; or R⁵ and an R³ are combined to form a C₃₋₆cycloalkyl, C₂₋₉heterocycloalkyl, C₆₋₁₀aryl, or C₂₋₉heteroaryl, wherein C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
R⁶, R⁷, R¹, and R⁹ are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15c};
R^{6a} is selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₁₋₉heteroaryl, —OR¹⁰, —SR¹⁰, —SF₅, —N(R¹⁰)(R¹¹), —C(O)OR₁₀, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, —N═S(═O)(R³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₁₀alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from R^{15c};
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₂₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹⁴ is independently selected from halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(RI), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{15d};
each R^{15a}, R^{15b}, R^{15c}, and R^{15d} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹¹, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹ l), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹I)—, —N═S(═O)(R¹³)₂, —S(═O)(═NH)N(R¹⁰)(R¹¹), —S(═O)(═NH)C(R¹⁰)(RI), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
m is 0, 1, 2, 3, 4, or 5; and
n is 0, 1, 2, 3, 4, 5, or 6;
and
(b) an additional agent,
wherein the combined amount of the compound of Formula (I) or Formula (II), or a pharmaceutically acceptable salt thereof and the additional agent is therapeutically effective for treating the cancer.

2. A method of treating cancer in a subject in need thereof, the method comprising administering to the subject:
(a) 5-methoxy-1-(2-methylpyridin-3-yl)-4-(prop-2-yn-1-ylamino)-7-(trifluoromethyl)quinazolin-2(1H)-one, 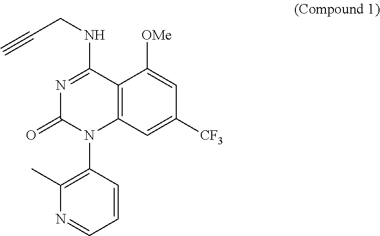 [SMILES: C#CCNc1nc(=O)n(-c2cccnc2C)c2cc(C(F)(F)F)cc(OC)c12]
or a pharmaceutically acceptable salt thereof; and
(b) an additional agent.

3. The method of claim 2, wherein the combined amount of Compound 1 or the pharmaceutically acceptable salt thereof and the additional agent are therapeutically effective.

4. The method of any one of claims 1-3, wherein the cancer is an MTAP-deficient cancer.

5. The method of any one of claims 1-3, wherein the cancer is an MTAP wild type cancer.

6. The method of any one of claims 1-5, wherein the cancer is a primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

7. The method of any one of claims 1-5, wherein the cancer is liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.

8. The method of any one of claims 1-7, wherein the additional agent is a PARP inhibitor, a CHK1 inhibitor, a MDM2 inhibitor, a hypomethylating agent, an mTOR inhibitor, an ATM inhibitor, a CDK 4/6 inhibitor, a BCL-2 inhibitor, a PRMT5 inhibitor, a PRMT1 inhibitor, an ATR inhibitor, a WEE1 inhibitor, an APE1 inhibitor, a topoisomerase inhibitor, a taxane, an immune checkpoint inhibitor, a CDK7 inhibitor, a CDK9 inhibitor, a DNA synthesis inhibitor, an antimetabolite, an AURORA inhibitor, a microtubule stabilizer, a DNA cross-linker, a vinca alkaloid, an alkylating agent, a PRMT6 inhibitor, a PRMT7 inhibitor, a PRMT9 inhibitor, a KRAS inhibitor, an EGFR inhibitor, a VEGFR inhibitor, an aromatase inhibitor, a mitotic inhibitor, a radiopharmaceutical agent, a cytotoxic agent, or any combination thereof.

9. The method of any one of claims 1-8, wherein the additional agent is a PARP inhibitor.

10. The method of claim 9, wherein the PARP inhibitor is olaparib (AZD2281), veliparib (ABT-888), rucaparib, talazoparib (BMN 673), AG-14361, INO-1001 (3-aminobenzamide), A-966492, PJ34 HCl, niraparib, UPF 1069, ME0328, RK-287107, pamiparib (BGB-290), NMS-P118, E7449, picolinamide, benzamide, NU1025, iniparib (B SI-201), AZD2461, BGP-15 2HC1, XAV-939, 4-hydroxyquinazoline, NVP-TNKS656, MN 64, or G007-LK, or a pharmaceutically acceptable salt thereof.

11. The method of claim 10, wherein the PARP inhibitor is olaparib (AZD2281), rucaparib, talazoparib (BMN 673), niraparib, or talazoparib (BMN 673), or a pharmaceutically acceptable salt thereof.

12. The method of any one of claims 1-8, wherein the additional agent is a hypomethylating agent.

13. The method of claim 12, wherein the hypomethylating agent is decitabine, azacitidine (5-azacytidine), RG108, thioguanine, zebularine, SGI-1027, CM272, 2′-deoxy-5-fluorocytidine, procainamide, bobcat339, gamma-oryzanol, thujaplicin, or (−)-epigallocatechin gallate, or a pharmaceutically acceptable salt thereof.

14. The method of claim 13, wherein the hypomethylating agent is procainamide or a pharmaceutically acceptable salt thereof.

15. The method of claim 13, wherein the hypomethylating agent is decitabine or a pharmaceutically acceptable salt thereof.

16. The method of any one of claims 1-8, wherein the additional agent is a mTOR inhibitor.

17. The method of claim 16, wherein the mTOR inhibitor is dactolisib (BEZ235), rapamycin (sirolimus), everolimus (RAD001), AZD8055, temsirolimus (CCI-779), PI-103, KU-0063794, torkinib (PP242), ridaforolimus (deforolimus, MK-8669), sapanisertib (MLN0128), voxtalisib (XL765), torin 1, torin 2, omipalisib (GSK2126458), OSI-027, PF-04691502, apitolisib (GDC-0980), GSK1059615, gedatolisib (PKI-587), WYE-354, vistusertib (AZD2014), WYE-125132 (WYE-132), PP121, WYE-687, WAY-600, ETP-46464, GDC-0349, XL388, GNE-477, bimiralisib (PQR309), SF2523, CZ415, paxalisib (GDC-0084), CC-115, onatasertib (CC 223), voxtalisib (XL765), zotarolimus (ABT-578), Tacrolimus (FK506), BGT226 maleate (NVP-BGT226 maleate), palomid 529 (P529), LY3023414 (samotolisib), biolimus-7, biolimus-9, azathioprine, campath 1H, or chrysophanic acid, or a pharmaceutically acceptable salt thereof.

18. The method of claim 17, wherein the mTOR inhibitor is everolimus (RAD001) or temsirolimus, or a pharmaceutically acceptable salt thereof.

19. The method of any one of claims 1-8, wherein the additional agent is a CDK 4/6 inhibitor.

20. The method of claim 19, wherein the CDK 4/6 inhibitor is palbociclib (PD-0332991), alvocidib, AT7519, JNJ-7706621, PHA-793887, BMS-265246, milciclib (PHA-848125), R547, riviciclib (P276-00), MC180295, G1T38, abemaciclib, ON123300, AT7519, purvalanol A, SU9516, ribociclib (LEE011), or BSJ-03-123, or a pharmaceutically acceptable salt thereof.

21. The method of claim 20, wherein the CDK 4/6 inhibitor is palbociclib (PD-0332991), or abemaciclib or a pharmaceutically acceptable salt thereof.

22. The method of any one of claims 1-8, wherein the additional agent is a BCL-2 inhibitor.

23. The method of claim 22, wherein the BCL-2 inhibitor is ABT-737, navitoclax (ABT-263), obatoclax (GX15-070), TW-37, venetoclax (ABT-199), AT101, HA14-1, sabutoclax, S55746, or gambogic acid, or a pharmaceutically acceptable salt thereof.

24. The method of claim 23, wherein the BCL-2 inhibitor is venetoclax (ABT-199) or a pharmaceutically acceptable salt thereof.

25. The method of any one of claims 1-8, wherein the additional agent is a PRMT5 inhibitor.

26. The method of claim 25, wherein the PRMT5 inhibitor is JNJ-64619178 (AGI-931), HLCL-61, GSK591, EPZ015666 (GSK3235025), GSK3326595 (EPZ015938; AGI-219), TNG908, TNG462, AMG193, AMG9747, MRTX1719, P305-05313, CTS3157, PH-020-803, or AZ-PRMT5i-1, or a pharmaceutically acceptable salt thereof.

27. The method of claim 26, wherein the PRMT5 inhibitor is TNG908, TNG462, AMG193, AMG9747, MRTX1719, or P305-05313, or a pharmaceutically acceptable salt thereof.

28. The method of any one of claims 1-8, wherein the additional agent is a WEE1 inhibitor.

29. The method of claim 28, wherein the WEE1 inhibitor is AZD1775 (MK1775), ZN-c3, debio 0123, IMP7068, SDR-7995, SDR-7778, NUV-569, PD0166285, PD0407824, SC-0191, DC-859/A, bosutinib, or Bos-I, or a pharmaceutically acceptable salt thereof.

30. The method of any one of claims 1-8, wherein the additional agent is a topoisomerase inhibitor.

31. The method of claim 30, wherein the topoisomerase inhibitor is epipodopyyllotoxin, SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, thiotepa, cyclosphosphamide, amsacrine, etoposide, etoposide phosphate, teniposide, daunorubicin, mitoxantrone, amsacrine, ellipticines, aurintricarboxylic acid, doxorubicin, or HU-331, or a pharmaceutically acceptable salt thereof.

32. The method of any one of claims 1-8, wherein the additional agent is an antimetabolite.

33. The method of claim 32, wherein the antimetabolite is 5-fluorouracil (5-FU), 6-mercaptopurine (6-MP), capecitabine, cytarabine, floxuridine, fludarabine, gemcitabine, hydroxy carbamide, methotrexate, pemetrexed, or phototrexate, or a pharmaceutically acceptable salt thereof.

34. The method of claim 33, wherein the antimetabolite is pemetrexed, 5-fluorouracil (5-FU), or pemetrexed, or a pharmaceutically acceptable salt thereof.

35. The method of any one of claims 1-8, wherein the additional agent is a microtubule stabilizer.

36. The method of claim 35, wherein the microtubule stabilizer is paclitaxel, nab-paclitaxel, docetaxel, colchicine, podophyllin, epothilone A, or epothilone B, or a pharmaceutically acceptable salt thereof.

37. The method of any one of claims 1-8, wherein the additional agent is a DNA cross-linker.

38. The method of claim 37, wherein the DNA cross-linker is oxaliplatin, cisplatin, or a pharmaceutically acceptable salt thereof.

39. The method of any one of claims 1-8, wherein the additional agent is a vinca alkaloid.

40. The method of claim 39, wherein the vinca alkaloid is vinorelbine, vincristine, vinblastine, vinblastine N-oxide, vindesine, vinflunine, vincamine, vintafolide, or deacetoxyvinzolidine, or a pharmaceutically acceptable salt thereof.

41. The method of any one of claims 1-8, wherein the additional agent is an alkylating agent.

42. The method of claim 41, wherein the alkylating agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin, platinum coordination complexes, or a pharmaceutically acceptable salt thereof.

43. The method of any one of claims 1-8, wherein the additional agent is a KRAS inhibitor.

44. The method of any one of claim 43, wherein the KRAS inhibitor is 6H05, adagrasib, ARS-1323, ARS-1323-alkyne, ARS-1620, ARS-1630, ARS-853, ASP2453, AZD4625, BAY-293, BI-0474, BI-2852, BI-3406, divarasib, G12Si-1, G12Si-5 formic, G12Si-5, garsorasib, K20, KRAS G12C inhibitor 1, KRAS G12C inhibitor 2, KRAS G12C inhibitor 3, KRAS G12C inhibitor 4, KRAS G12C inhibitor 5, KRAS G12C inhibitor 13, KRAS G12C inhibitor 14, KRAS G12C inhibitor 15, KRAS G12C inhibitor 16, KRAS G12C inhibitor 17, KRAS G12C inhibitor 18, KRAS G12C inhibitor 23, KRAS G12C inhibitor 24, KRAS G12C inhibitor 25, KRAS G12C inhibitor 26, KRAS G12C inhibitor 27, KRAS G12C inhibitor 28, KRAS G12C inhibitor 32, KRAS G12C inhibitor 43, KRAS G12C inhibitor 44, KRAS G12C inhibitor 45, KRAS G12C inhibitor 46, KRAS G12C inhibitor 47, KRAS G12C inhibitor 48, KRAS G12C inhibitor 49, KRAS G12C inhibitor 50, KRAS G12C inhibitor 51, KRAS G12C inhibitor 52, KRAS G12C inhibitor 53, KRAS G12C inhibitor 54, KRAS G12C inhibitor 55, KRAS G12C inhibitor 57, K-Ras G12C-IN-2, KRAS G12D inhibitor 3, KRAS G12D inhibitor 7, KRAS G12D inhibitor 14, KRAS G12D inhibitor 16, KRAS G12D inhibitor 17, KRAS inhibitor-3, KRAS inhibitor-6, KRAS inhibitor-7, KRAS inhibitor-8, KRAS inhibitor-10, KRAS inhibitor-11, KRAS inhibitor-12, KRAS inhibitor-13, KRAS inhibitor-14, KRAS inhibitor-15, KRAS inhibitor-16, KRAS inhibitor-17, KRAS inhibitor-18, KRAS inhibitor-20, K-Ras(G12C) inhibitor 6, KRpep-2d, LC-2, MRTX1133, MRTX-1257, MRTX849 acid, MRTX-EX185 formic, opnurasib, Pan KRas-IN-1, PROTAC K-Ras Degrader-1, RM-018, SAH-SOS1A, SOS1-IN-4, SOS1-IN-9, sotorasib, or ZG1077, or a pharmaceutically acceptable salt thereof.

45. The method of any one of claim 44, wherein the KRAS inhibitor is adagrasib, divarasib, garsorasib, opnurasib, or sotorasib, or a pharmaceutically acceptable salt thereof.

46. The method of any one of claim 45, wherein the KRAS inhibitor is sotorasib or a pharmaceutically acceptable salt thereof.
